(19) United States
(12) Patent Application Publication
(10) Pub. No.: US 2005/0042307 A1
(43) Pub. Date: Feb. 24, 2005

(54) Gel-forming polysaccharide from psyllium seed husks

(76) Inventors: Judith A. Marlett, Sun City, AZ (US); Milton H. Fischer, Middleton, WI (US)

Correspondence Address: WOODCOCK WASHBURN LLP, ONE LIBERTY PLACE, 46TH FLOOR, 1650 MARKET STREET, PHILADELPHIA, PA, 19103, US

(21) Appl. No.: 10/848,595

(22) Filed: May 17, 2004

Related U.S. Application Data
(63) Continuation-in-part of application No. 10/009,097, filed on May 21, 2002.

(51) Int. Cl. A61K 31/715; A61K 35/78; C08B 37/00

(52) U.S. Cl. 424/738; 514/54; 536/123

(57) ABSTRACT

A purified fraction of the gel-forming component of psyllium seed husks is disclosed, along with process for purification and use of the fraction. The partially purified composition disclosed comprises an arabinoxylan compound comprising a highly branched backbone of β 1,4 linked xylopyranose residues with branches predominantly of xylose, or a trisaccharide consisting of arabinose-xylose-arabinose. The compounds form digestion resistant gels and have laxative and cholesterol-reducing effects.

### CROSS REFERENCE TO RELATED APPLICATIONS

This is a continuation in part of U.S. application Ser. No. 10/009,097, filed May 21, 2002 which is based on PCT/US00/15693, filed Jun. 8, 2000, which claims priority to U.S. application Ser. No. 09/328,611, filed Jun. 9, 1999, now U.S. Pat. No. 6,287,609, the entireties of which are incorporated herein by reference.

### FIELD OF THE INVENTION

This invention relates to the field of laxatives, treatments to lower blood serum cholesterol and low-calorie food thickeners and fat substitutes. In particular, the invention relates to unfermented gel-forming polysaccharides from psyllium seed husks, their characterization, and methods for their isolation.

### BACKGROUND OF THE INVENTION

Various scientific and scholarly articles are referred to throughout the specification, for example by number in parentheses. These articles are incorporated by reference herein to describe the state of the art to which this invention pertains.

The seed husks of psyllium (Plantago ovata, also known as ispaghula) are commonly used as a laxative and to promote regular bowel function. Psyllium seed husks promote Taxation partially by increasing the mass and moisture content of the stool (Marteau et al., 1994, Gut 35:1747-1752). Additionally, the excreta of animals and humans fed diets containing psyllium seed husk is gelatinous. This gelatinous property contributes to laxative properties of psyllium seed husks by decreasing friction in the gut. Observed increases in fecal mass and water retention also have been attributed to this gelatinous material (Narteau et al., 1994, supra). The gel is composed largely of unfermented psyllium polysaccharides (Cabotaje et al., 1994, 1302-1307).

Psyllium seed husk has many of the properties of soluble dietary fiber sources. Commonly used sources of soluble dietary fiber include pectin, gums and oat bran. Soluble dietary fiber (SDF) has many uses in food and medicinal preparations. Soluble fibers are components of minimally processed food sources such as oats, oat bran and barley, or are available as concentrates, such as gums, pectins and mucilages. Gums and mucilages are carbohydrate polymers that are generally isolated from plant sources. Mucilages in particular produce slippery or gelatinous solutions in water. Pectins are polymeric chains of partially methylated galacturonic acids that also possess the ability to form a gel in water. Most soluble fibers are rapidly and completely fermented and have no Taxation properties.

Sources of soluble dietary fiber that are also viscous lower serum cholesterol in animals and humans (Marlett, 1997, pp. 109-121, Dietary Fiber and Health, Plenum Press, New York, ed. Kritchevsky and Bonfield). The viscosity of the SDF, rather than its fermentation in the gastrointestinal tract, is key to its hypocholesterolemic action (Marlett et al., 1994, Hepatology 20:1450-1457). Viscosity in the lumen of the lower small intestine interferes with the absorption of bile acids and more bile acids are lost through the stool. Blood cholesterol is thought to be lowered primarily because it is being used in the liver to synthesize more bile acids to replace those lost. The synthesis of bile acids in the liver accounts for 40 to 50% of the daily elimination of cholesterol from the blood. However, the addition of one source of soluble fiber, oat bran, to the diet also increases the proportion of deoxycholic acid in the bile acid pool, which decreases the absorption of exogenous dietary cholesterol. Supplementing the diet with psyllium husk also increases the excretion of bile acids by about 50% (Gelissen et al., 1994, Am. J. Clin. Nutr. 59:395-400).

Soluble dietary fiber concentrates are also often used as thickeners and low calorie fat substitutes in the food industry because of their hydrocolloidal properties (Ward, 1997, Cereal Foods World, 42:386-390). Low-viscosity gums such as gum acacia have both hydrophilic and lipophilic properties that make them ideal as emulsifiers, surfactants and stabilizers. Pectins and mucilages have gel-forming properties that made them ideal thickeners of food products. Pectins are traditionally extracted from apple and citrus fruits. Commonly used mucilages are generally extracted from seaweed and include carrageenan, agar and alginate. A fat substitute can be made by combining gum with mucilage and/or pectin to create a compound with the emulsifying properties and smoothness of a fat.

Currently used preparations of psyllium seed husks have certain disadvantages. Laxative preparations of psyllium seed husks are generally composed of ground husk and have coarse and unpleasant mouthfeel when administered in drinks. Psyllium seed husks have been incorporated into cookies, crackers and similar products; however, these products have a tendency to begin to gel unpleasantly in the mouth. More significantly, though, psyllium seed husks can swell in the esophagus, producing an esophageal obstruction that can cause choking. For this reason, psyllium seed husk preparations are not recommended for ingestion by persons who may have difficulty swallowing (e.g., elderly persons). Finally, the recommended daily dose of psyllium husk of 3.5-11 g per day is inconvenient to ingest in any form.

Given the many beneficial properties of pysllium seed husk, a better understanding of the chemical structure of the husk component(s) responsible for the these properties has been sought. In early chemical studies, Laidlaw and Percival (3, 4) analyzed the polysaccharide mucilage extracted from whole seeds by first cold, then hot water. They secured evidence for two components, which they characterized as a polyuronide and a neutral arabinoxylan. Later Kennedy and coworkers (5, 6) studied the mucilage obtained from Plantago seed husk by extraction with alkali, and concluded that the preparation, although polydisperse, represented a single species of polysaccharide, a highly branched, acidic arabinoxylan.

Marlett and Fischer (2002) developed an efficient, reproducible process for the alkaline extraction and fractionation of the polysaccharide from the husk in order to obtain material for biological studies, and to address issues relating to the composition and structure of the active substance (7). Through animal and human feeding experiments they established that a gel-forming fraction, amounting to some 55-60% of the husk, is responsible for both the laxative and cholesterol-lowering activities (7, 8).

Other viscous, non-nutrient polysaccharides, such as β-glucans and pectins, lower blood cholesterol levels by the same mechanism as psyllium (9) but these substances have negligible effects on bowel function. They are rapidly and completely fermented in the gut, whereas psyllium husk largely survives, increasing stool output and imparting a gel-like consistency to the excreta (8, 10).

Determinations of the chemical composition and physical structure of the active fraction of psyllium mucilage are needed in the art to be able to better understand the chemical and physical properties required for a form of psyllium husk that is convenient and pleasant to use.

### SUMMARY OF THE INVENTION

The present invention provides the gel-forming component of psyllium seed husks in a purified form. This gel fraction provides the Taxation and hypocholesterolemic effects of intact psyllium seed husks, but is in a form that is easily administrable as a tablet, capsule or liquid, without certain unpleasant or unsafe qualities associated with the use of intact psyllium seed husks. The gel fraction also has utility in treatment of other intestinal abnormalities and maintaining normal bowel function, and as a food thickener and fat replacement.

According to one aspect of the invention, a gel-forming fraction of psyllium seed husks that survives microbial fermentation upon passage through a monogastric mammalian digestive tract is provided. Among other components, the gel fraction comprises predominantly xylose and arabinose in a dry weight ratio of at least about (preferably about 3.5). The fraction comprises notably limited amounts of other sugars, e.g., about 2.5% -13.5% total of rhamnose, galactose, glucose and uronic acids. More specifically, the gel-forming fraction has the following sugar composition, expressed as a percentage of total sugars:
between about 0.5% and 4% rhamnose;
between about 19% and 22% arabinose;
between about 68% and 76% xylose;
between about 0% and 0.5% mannose;
between about 1% and 2% galactose
between about 0% and 1% glucose; and
between about 1% and 6% uronic acids

Upon further purification, the gel-forming fraction becomes even more depleted in rhamnose, glucose and uronic acids.

The gel-forming fraction is also highly viscous, a 0.2% concentration in formamide having an apparent viscosity of at least 500 sec, preferably 750 sec, and most preferably 850 sec. The fraction is soluble in a dilute alkaline solution and forms a gel upon acidification of the solution to a final pH of about 4.5.

According to another aspect of the invention, in a preferred method of obtaining the psyllium seed husk gel-forming fraction, a separate carbohydrate fraction is also obtained. This fraction is soluble in the dilute alkaline solution and remains soluble upon acidification of the solution to a pH of about 4.5. This fraction is comprised of xylose and arabinose in a ratio of at least about 4:1 and further comprises at least about 12% (by weight) rhamnose and at least about 15% (by weight) uronic acids.

According to another aspect of the invention, a method of fractionating psyllium seed husks to obtain a gel-forming fraction and an additional carbohydrate fraction is provided. The method comprises: (a) mixing the husks, in the presence of a chemical reducing agent, in an aqueous alkaline solution comprising between about 0.15 and about 1.0 M (preferably 0.15-0.5 M, more preferably 0.15-0.4 M, even more preferably 0.15-0.3 M and most preferably 0.15-0.2 M) hydroxyl ions, thereby fractionating the husks into an alkali soluble fraction and an alkali-insoluble fraction; (b) removing the alkali insoluble fraction; (c) acidifying the alkali soluble fraction to a pH of between about 3 and about 6 (preferably between about 4 and about 5, most preferably about 4.5), which results in the gelation of the gel-forming fraction; and (d) separating the gel fraction, e.g., by centrifugation or other means, from the additional carbohydrate fraction contained in the acidified solution. In preferred embodiments, the method further comprises washing the gel fraction with an aqueous or buffered solution and desiccating the washed gel fraction.

In another aspect of the invention, a gel-forming fraction from psyllium seed husks is provided, which is produced by the aforementioned procedure. The additional carbohydrate fraction is also provided in this aspect of the invention.

According to another aspect of the invention, alternative methods are provided for obtaining a polysaccharide fraction that contains the aforementioned gel-forming fraction. Such a fraction is obtained by solvent extraction using formamide, dimethylsulfoxide or 4-methylmorpholine N-oxide (50% solution in water). The solvent-treated material is centrifuged to recover the soluble materials that are then poured into ethanol to achieve a concentration of 80% ethanol. The precipitate that forms is similar in composition to the alkali-soluble gel-forming fraction.

According to another aspect of the invention, pharmaceutical preparations for treatment of constipation or other intestinal abnormalities, or for lowering serum cholesterol levels in a patient are provided. These preparations are formulated to contain effective dosages of the psyllium seed husk gel-forming fraction of the invention. Methods of treating patients for these various conditions are also provided, which comprise administering the pharmaceutical preparations of the invention.

In another aspect of the invention, a composition of Formula I is provided, comprising a backbone β 1,4 linked xylopyranose residue wherein K¹, K², K³, and K⁴ are each independently selected from hydrogen, xylose, or a trisaccharide consisting of arabinose-xylose-arabinose, and in some instances galactose; and Z1 and Z2 are each preferably hydrogen.

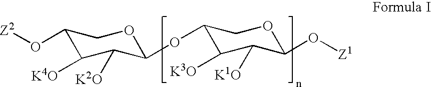

The compounds are able to form gels in water. A solution of 0.5% or greater is a rigid gel at neutral or acidic pH and room temperature. At pH greater than about 10, the gelling properties are substantially diminished or lost. The compounds retain their gel-forming capacity even after passage through the gut, and substantially resist fermentation by the intestinal microflora.

In another aspect of the invention a gel-forming polysaccharide is provided. The polysaccharide comprises a backbone structure of β 1,4 linked xylopyranose residues. At least 50% of the backbone xylose residues have branches at of the 0-2 and 0-3 positions, said branching structures comprising xylose or a trisaccharide of Ara-Xyl-Ara. The composition of the polysaccharide is at least about 70 percent xylose and 20 percent arabinose, of which substantially all of the xylose residues are in the pyranose ring form and substantially all of the arabinose residues are in the furanose form.

In another aspect of the invention a laxative polysaccharide comprising an arabinoxylan is provided, the polysachharide containing at least about 20% arabinose and 70% xylose. Acid hydrolysis of the polysaccharide results in the release of some monsaccharides such that after 2 hours of acid hydrolysis the composition of the partially digested polysaccharide was less about 20 percent arabinose and greater than about 75% xylose; after 6 hours of acid hydrolysis, the partially digested polysaccharide comprises less than about 15% arabinose and greater than about 80% xylose.

Other features and advantages of the present invention will become apparent from the detailed description and examples that follow.

### BRIEF DESCRIPTION OF THE DRAWINGS

FIG. 1: Representative HMQC and HMQC-TOCSY spectra from the NMR analysis Each set of ¹H—¹³C correlation peaks (identified as such from the usefully redundant HMQC-TOCSY correlations) has a different color, and the major sets are further defined by grid lines.

Panel A: HMQC spectrum of fraction B in DMSO is deceptively clean. Arabinofuranose peaks (blue).

Panel B: The spectrum of the fraction solubilized (B-E2) by the arabinofuranosidase treatment differs from that of the starting material only by the complete disappearance of the arabinofuranose peaks.

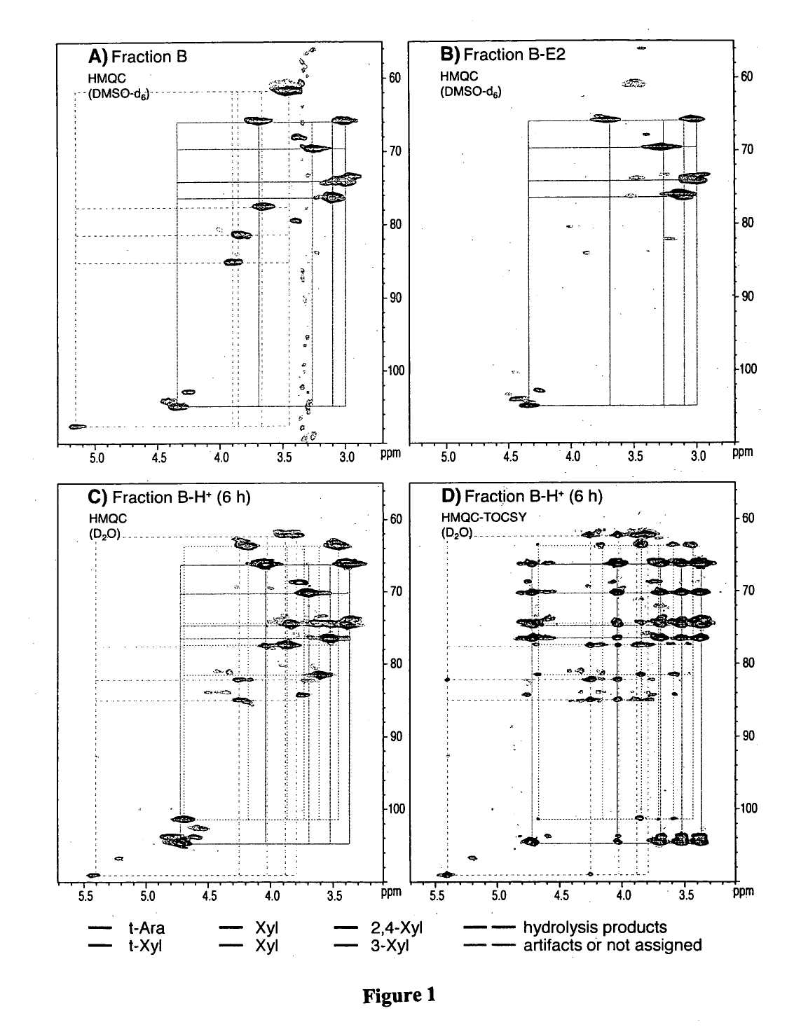

Panels C and D: The 6 h acid hydrolysis product, which formed a low-viscosity solution, gave more detailed spectra (HMQC, FIG. 1C; HMQC-TOCSY, FIG. 1D), determined in D₂O.

### DETAILED DESCRIPTION OF ILLUSTRATIVE EMBODIMENTS

The present invention provides a highly polymerized gel-forming fraction from psyllium seed husks that has great utility in the treatment and prevention of constipation and as a hypocholesterolemic agent. This gel fraction remains substantially unfermented during transit through the gastrointestinal tract (Example 6 and Example 7) and promotes Taxation through a variety of means, including increasing moisture content and overall mass of stool and imparting to the stool a slippery characteristic that facilitates ease of passage of stool (Example 6).

The gel-forming fraction from psyllium seed husks has also been demonstrated as a hypocholesterolemic agent. Accordingly, the gel-forming fraction may be used alone or in combination with other active substances as a therapeutic treatment to lower serum cholesterol.

Stool from rats and humans fed psyllium seed husks are sometimes gelatinous (Cabotaje et al., 1994, supra; Example 6). However, attempts to purify and characterize a gel-forming material (Kennedy et al., Carbohydrate Res. 75:265-274, 1979; Sandhu et al., Carbohydrate Res. 93:247-259, 1981) have not been successful and none has provided the highly purified gel-forming fraction having the features described in accordance with the present invention. The method of fractionating psyllium seed husks developed in accordance with the invention has led to the unexpected discovery, contrary to published reports (e.g., Kennedy et al., 1979, supra), that a gelatinous, alkali-soluble fraction of psyllium seed husks can be further fractionated to form a highly viscous gel fraction (referred to herein as “Fraction B”) and a second carbohydrate fraction with distinctive compositional features, as described in greater detail below (referred to herein as “Fraction C”). Both the viscous, gel-forming Fraction B and the additional Fraction C are soluble in a variety of substances, including dilute and concentrated alkali, formamide, dimethysulfoxide and 4-methylmorpholine N-oxide (50% aqueous solution); consequently, these two fractions together can be isolated on the basis of these solubility characteristics. However, the further separation of the fractions has been either unsuccessful (e.g., a strong alkali-extracted gel-forming fraction was unable to be further separated by Kennedy et al., 1979; supra) or has remained unexplored. The present inventors have discovered that, using a suitable first extraction procedure to obtain a product comprising fractions B and C together, that the fractions can be separated by acidification of a solution containing a mixture of the fractions. Fraction B is concentrated for separation from the acidified mixture by centrifugation, whereas Fraction C remains soluble in the acid.

Description of Carbohydrate Polymers

The viscous, gel-forming psyllium seed husk fraction of the invention (Fraction B) is comprised, in one embodiment, primarily of xylose and arabinose. In a preferred embodiment, the gel-forming fraction has at least 50% xylose and arabinose by weight, in a more preferred embodiment at least 75% xylose and arabinose by weight, and in a most preferred embodiment at least 85% xylose and arabinose by weight. The gel-forming fraction in formamide at 0.02% has an efflux time, as determined by the method in Example 3, of at least 500 sec as measure in an Ostwald viscometer. In a more preferred embodiment the efflux time is at least 750 sec, and in a most preferred embodiment, at least 850 sec. The gel-forming fraction is furthermore particularly deficient in rhamnose, galactose and uronic acids, as compared to xylose. In a preferred embodiment, the ratio of weights of xylose to rhanmose is more than 50, in a more preferred embodiment the ratio is more than 60, and in a most preferred embodiment, the ratio is more than 65. In a preferred embodiment, the ratio of weights of xylose to galactose is more than 25, in a more preferred embodiment the ratio is more than 35, and in a most preferred embodiment, the ratio is more than 42. In a preferred embodiment, the ratio of weights of xylose to uronic acid is more than 15, in a more preferred embodiment the ratio is more than 25, and in a most preferred embodiment, the ratio is more than 35. In a preferred embodiment, the ratio of weights of xylose to arabinose of Fraction B is between 2.5 and 4.5, in a more preferred embodiment the ratio is between 3.0 and 4.0, and in a most preferred embodiment, the ratio is between 3.25 and 3.75.

The acid-soluble psyllium seed husk fraction (Fraction C) is also high in xylose and arabinose. In a preferred embodiment, the acid-soluble fraction has at least 25% xylose and arabinose by weight, in a more preferred embodiment at least 40% xylose and arabinose by weight, and in a most preferred embodiment at least 45% xylose and arabinose by weight. Though Fraction C has an apparent viscosity similar to that of fraction B, it does not have the gel-forming property of Fraction B. Fraction C is furthermore particularly enriched in rhamnose, galactose and uronic acids, as compared to xylose. In a preferred embodiment, the ratio of weights of xylose to rhamnose is less than 6.0, in a more preferred embodiment the ratio is less than 4.5, and in a most preferred embodiment, the ratio is less than 3.0. In a preferred embodiment, the ratio of weights of xylose to galactose is less than 40, in a more preferred embodiment the ratio is less than 30, and in a most preferred embodiment, the ratio is less than 25. In a preferred embodiment, the ratio of weights of xylose to uronic acid is less than 30, in a more preferred embodiment the ratio is less than 10, and in a most preferred embodiment, the ratio is less than 5.0 In a preferred embodiment, the ratio of weights of xylose to arabinose of Fraction C is more than 3.0, in a more preferred embodiment the ratio is more than 4.0, and in a most preferred embodiment, the ratio is more than 4.5.

Preparation of Psyllium Seed Husk Fractions

The present invention also provides methods of fractionating psyllium seed husks to yield the purified and separated fractions described above. In its most basic form, the method has the following steps:
1. Suspend psyllium seed husks in a dilute alkaline aqueous solution (preferably 0.15-0.2 M, hydroxyl ions) containing a reducing agent, in which portions of the husk material will dissolve, while a certain portion remains insoluble;
2. Remove the alkaline-insoluble material (referred to herein as “Fraction A”), e.g., by centrifugation;
3. Acidify the alkali-soluble fraction of step one to a pH of between 3 and 6, preferably 4.5, to yield a gel (Fraction B) and an acid-soluble fraction (Fraction C); and
4. Separate the gel from the acidified solution, e.g., by centrifugation.

One example of this method is taught in Example 1. Many variations exist to the method that will not substantially change the product isolated. These are described in detail below.

The alkaline solubilization step has several variations. The method taught in Example 1 has improved this solubilization over that found in the prior art. Previous alkaline solubilizations of psyllium seed husk polysaccharides utilized concentrated solutions of base (i.e. 1.2 M NaOH, Kennedy et al., 1979, supra) without a reducing agent. Recognizing the harsh nature of this treatment and its partial degradation of polysaccharide chains in the gel-forming fraction, the inventors have demonstrated that a gel-forming fraction can be obtained, presumably in a form more suitable for further fractionation, using a much less concentrated alkaline solution and a suitable chemical reducing agent, such as borohydride. Though up to 4 N alkaline solution can be utilized, the concentration of base in the alkaline solubilization is preferably at least 0.15 N and not more than 1.0 N; in a more preferred embodiment, at least 0.15 N and not more than 0.5 N; and in the most preferred embodiment, at least 0.15 N and not more than 0.2-0.3 N. Any standard base can be used in the alkaline extraction, including, but not limited to, sodium hydroxide, potassium hydroxide, lithium hydroxide, ammonium hydroxide and tetramethyl ammonium hydroxide.

A chemical reducing agent, such as borohydride, should be added to the alkaline solubilization step to minimize base-catalyzed depolymerization. In Example 1, a concentration of 1 g/L of sodium borohydride is used, but effective concentrations range from about 50 mg/L to 10 g/L. In a preferred embodiment, the sodium borohydride concentration is at least 100 mg/L and not more than 4 g/L, in a more preferred embodiment at least 500 mg/L and not more than 2 g/L, and in a most preferred embodiment, at least 800 mg/L and not more than 1.2 g/L. Other forms of borohydride also are suitable for use in this step, including but not limited to, lithium borohydride, potassium borohydride and sodium cyanoborohydride.

The degree of initial processing of psyllium seed husks may alter the alkaline solubilization in ways that will be well known to those skilled in the art. It is important that the husk material be processed so that it is in small pieces, in order to allow the viscous polysaccharides to be easily separated from the insoluble and fibrous materials of the cell walls. In Example 1, the psyllium seed husks are milled, but any process that pulverizes the plant material can be used, and these processes are well known in the art.

The ratio of seed husk material to alkaline solution can be important for efficient solubilization of the polysaccharide fractions. In Example 1, a ratio of 2 g psyllium seed husk is added to 400 ml of alkaline solution, but this ratio can be varied without appreciably affecting the solubilization. For instance, the ratio can be varied so as to add as little as about 0.1 g seed husks or as much as about 4 g seed husks to a 400 ml alkaline solution. Additionally, the time of solubilization can be varied to optimize the procedure (0.5 hr-24 hr) at a range of temperatures (4-50° C.).

Step two of the method of the invention requires that the alkaline insoluble materials be separated from the alkaline soluble materials. In Example 1, centrifugation is employed to accomplish this objective. However, numerous variations and other procedures may be substituted without substantially changing the soluble materials isolated. One skilled in the art will know how to alter the time and force of the centrifugation to adapt the separation to different centrifuge rotors, plant materials and alkaline solutions. Other methods that will accomplish this separation are well known in the art. Some of these methods will be better suited to large scale use of the method of the invention. Separation methods of interest include, but are not limited to, flow-through centrifugation or filtration (with agitation). Example 1 further teaches washing the insoluble materials with the alkaline solution and re-separation to improve the yield of the alkaline soluble materials. This washing step is optional but can be used to advantage to improve yield.

Step three requires that the alkaline soluble materials of step two be subjected to acidification. In Example 1 this is accomplished by adding glacial acetic acid to the combined alkaline soluble materials until the pH is adjusted to 4.5. The range of pH used for this acid solubilization can be varied without substantial effect on the products. In a preferred embodiment, the pH is between 3 and 6, in a more preferred embodiment, the pH is between 4 and 5, and in a most preferred embodiment, the pH is about 4.5 as described in Example 1. The choice of acid is also subject to variation. Examples of acids suitable for use in this step are acetic, hydrochloric, sulfuric, oxalic, trichloroacetic and trifluoroacetic acids, among others. Here as in step one, the duration, temperature, etc. of the solubilization can be varied, but preferably is carried out at ambient temperature for about 2 hours.

Step four requires that the acid insoluble gel-like material (Fraction B) be separated from the acid soluble materials (Fraction C). Centrifugation is typically employed to accomplish this separation. An optional washing of the insoluble gel mass (e.g., with water, buffer or other suitable solvent) may also be performed to improve the efficiency of the separation.

As alternatives to centrifugation in step four, two other approaches, both of which are amenable to large scale preparations, may be utilized. The inventors have observed that gel-like material of Fraction B floats, and that the gel has good integrity. Accordingly, the gel can be strained or skimmed from the acidified mixture using a paddle, for instance, such as the type used to remove curds during cheese-making. The gel material is thereafter deposited into a separate container, where it may be washed as a further purification step. Alternatively, the vessel containing the acidified solution with the gel floating on top can be drained from the bottom by gravity or by gentle vacuum, to leave the gel at the bottom of the vessel. Again, the gel may be washed.

Preparing Fraction B and Fraction C for storage and/or use may employ several procedures. The polysaccharide preparations of Fractions B and C may be used or stored hydrated. If stored in a hydrated form, preservatives or bacteriostatic agents may be added. Drying the polysaccharide preparations is particularly advantageous for use or storage. In a preferred embodiment, Fraction B and Fraction C are desiccated by treatment with 95% ethanol, washing with diethyl ether and drying. The fractions also may be desiccated with other solvents, such as methanol, acetone or isopropyl alcohol. Any standard dehydration method (e.g., evaporation, lyophilization) may be used to dry the fractions, provided the temperature is maintained at less than about 60° C., more preferably at less than about 400° C.

Uses of Psyllium Seed Husk Fractions

The psyllium seed husk fractions of the invention have uses as therapeutic treatments. In this regard, the viscous, gel-forming fraction, Fraction B, has been demonstrated effective in promoting Taxation and also as a hypocholesterolemic agent. This material can be used alone or in combination with other active substances in therapeutic or prophylactic preparations for constipation, diarrhea and/or high serum cholesterol. Such preparations can incorporate the gel-forming fraction in pills, capsules or liquids to be administered by mouth. In a preferred embodiment, a dried form of the gel is formulated for convenient administration as a pill or capsule. The gel re-hydrates upon ingestion. With regard to re-hydration, the gel-forming fraction possesses hydration characteristics that are particularly advantageous. After the gel-forming fraction has been isolated and dried, it is slow to hydrate. However, based on observations following feeding the material to colectomized rats, it is clear that the gel does become hydrated in the upper gut, where it exerts its hypocholesterolemic effect. This delay in hydration is advantageous inasmuch as the risk of premature hydration, e.g., by a delay in the esophagus, is minimized or avoided.

The gel-forming preparations can additionally be incorporated into food products. Since the active polysaccharides have been isolated away from the other plant cell components by the method of the invention, they will not have the unpleasant mouthfeel or the necessity to administer large dosages associated with the psyllium seed husk preparations currently in use.

The unfermented gel-forming polysaccharide from psyllium seed husks is well-known for its laxative effects on the monogastric mammalian digestive tract. For an adult human, a suitable dosage of the gel-forming fraction in dry form is about 2 g, one to three times a day, to maintain bowel regularity and as a treatment for constipation.

As described in Example 4, the gel-forming fraction of psyllium seed husks has been demonstrated as a hypocholesterolemic agent. Accordingly, this fraction also may be used alone or in combination with other active substances as a therapeutic treatment to lower serum cholesterol. For an adult human, a suitable dosage of Fraction B in dry form is from about 3 g to about 7 g daily. The psyllium husk Fractions B and C of the invention can additionally be used as food additives. They may be used as thickeners, gel-formers and bulking agents in prepared foods. They may also be combined with other food additives to make fat mimetic systems. Because the polysaccharide preparations of the invention are partially non-digested, they will additionally be low calorie, serum cholesterol-lowering and laxation-promoting. The polysaccharide preparations of Fractions B and C can be used in many of the food products where gums and mucilages are currently used.

Further Characteriziation of the Gel-Forming Fraction

In another aspect of the invention, a compound of Formula I is provided comprising a backbone β 1,4 linked xylopyranose residue wherein K¹, K², K³, and K⁴ are each independently selected from hydrogen, xylose, or a trisaccharide consisting of arabinose-xylose-arabinose; and Z¹ and Z² are each preferably hydrogen. Occassionally, in some embodiments K¹, K², K³, and K⁴ may each independently be galactose.

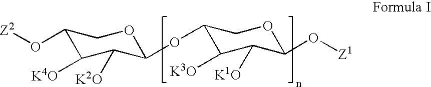

In certain preferred embodiments, K¹ and K² are each independently selected from hydrogen or xylose and K³ and K⁴ are each independently selected from hydrogen or a trisaccharide consisting of Arabinose-Xylose-Arabinose. The residues in the trisaccharide for branching are bonded through the anomeric carbon. Most preferably, the trisaccharide has the structure Ara(α1→3)-Xyl(β1→3)-Ara(α1→, or even L-Araf(α1→3)-D-Xylp(β1→3)-L-Araf(α1→.

In preferred embodiments, the arabinose and xylose are present as ring structures. More preferred are embodiments wherein the arabinose residues are each in a furanose ring and the xylose residues are each in a pyranose ring, thus the polysaccharide comprises xylopyranose residues and arabinofuranose residues.

The backbone of the compound is highly branched. Preferred embodiments provide the compound of claim 1 wherein at least 50% of the total K¹, K², K³, and K⁴ are each independently selected from xylose and the trisaccharide Ara-Xyl-Ara, with an occasional galatose residue. More preferred are compound wherein at 66% of the total K¹, K², K³, and K⁴ are each independently selected from xylose and the trisaccharide Ara-Xyl-Ara. Still more preferred are compounds wherein at least 75% of the total K¹, K², K³, and K⁴ are each independently selected from xylose and the trisaccharide Ara-Xyl-Ara. Even more preferred are those compounds having at least 80% of the total K¹, K², K³, and K⁴ each independently selected from xylose and the trisaccharide Ara-Xyl-Ara. Still more preferred are certain compounds wherein about 85% of the total K¹, K², K³, and K⁴ are each independently selected from xylose and the trisaccharide Ara-Xyl-Ara. Galactose residues may occasionally comprise the K¹, K², K³, and K⁴ positions in each of these embodiments. Preferably less than 10% of the K¹, K², K³, and K⁴ are galactose. More preferably, less than 8, 6, 5, 4, 3, 2 or 1% of the residues at positions are galactose residues.

Inherently, highly branched polysaccharides have a number of branching points approximately equal to the number of terminal residues present, in the instant invention, preferred compounds have about one-third of the residues in branching positions. In some preferred embodiments, at least about 30% of the glycosyl residues of the compound are in nonreducing terminal positions. More preferred are those compounds with 32, 33 and 34% of the residues in terminal positions, as well as those with 35 and 36% of the residues in terminal positions to allow for certain doubly branched residues.

Preferably, the compounds form gels in water. Gels are most easily formed in acidic solutions. Preferred pH for gel-formation is less than 10, above which the gels are lost. A solution of 0.5% or greater is a rigid gel at neutral or acidic pH and room temperature. The compounds retain their gel-forming capacity even after passage through the gut, and substantially resist fermentation by the intestinal microflora.

The compounds of the invention preferably resist digestion by intestinal microflora as determined by standard methods, such as those set forth in Examples 6 and 7.

Preferred compounds comprise at least about 70% xylose and 19% arabinose, and not exceeding 2% galactose. Still more preferred are those compounds which have at least about 72% xylose and 20% arabinose. More highly preferred compounds have at least about 74% xylose and 22% arabinose. Minor amounts of other components, including but not limited to galactose, glucose, rhamnose and uronic acid may be detected in certain compounds of the invention.

The compounds, in certain presently preferred embodiments can be isolated from plants. Particularly preferred presently are compounds wherein the plant is Plantago ovata. Plantago ovato Forsk is a presently preferred variety of Plantago.

The compounds in preferred embodiments have a laxative effect in mammals, most preferably humans. In other embodiments the compound possesses cholesterol-lowering activity in mammals, in particular, in humans.

In preferred embodiments, the compound has a MW greater than about 6,000 da and is 100% retained by a membrane with a nominal pore size which retains molecules greater than about 6,000-8,000 da. The compounds are able to form gels in water under conditions of neutral to acidic pH, at 0.5% concentration. Solutions of the compounds are viscous even after passage through the gut. The compounds also resist fermentation by the intestinal microflora, as determined by the methods described in Examples 6 and 7.

In another aspect of the invention a gel-forming polysaccharide is provided. The polysaccharide preferably comprises a backbone structure of β 1,4 linked xylopyranose residues. At least 50% the backbone xylose residues have branches at the O-2 and O-3 positions. The branching structures preferably comprise xylose, or an arabinoxylan trisaccharide. In preferred embodiments the trisaccharide is Ara-Xyl-Ara. The composition of the polysaccharide is at least about 70 percent xylose and 19 percent arabinose. Substantially all of the xylose residues are in the pyranose ring form and substantially all of the arabinose residues are in the furanose form.

In preferred embodiments, the highly branched polysaccharide has greater than 50% of its O-2 and O-3 backbone positions substituted with xylose or the trisaccharide. In more preferred embodiments at least about 60, 70, 75, 80, 81, 82, 83, 84 or 85% of the O-2 or O-3 positions are branching points comprising either an xylose residue or a Ara-Xyl-Ara trisaccharide. In more preferred embodiments, the trisaccharide branch has the structure Ara(α1→3)-Xyl(β1→3)-Ara(α1→, or even the even more preferred L-Araf(α1→3)-D-Xylp(β1→3)-L-Araf(α1→.

In presently preferred embodiments, approximately one third of the total glycosyl residues in the polysaccharide are in nonreducing terminal positions. Also preferred are polysaccharides in which the proportion of unsubstituted (1→4) xylose is relatively small.

The gel-forming polysaccharide preferably comprises xylose residues in the xylopyranose form and arabinose residues in the arabinofuranose form. Most preferably there are no xylofuranose or arabinopyranose residues in the polysaccharide. In preferred embodiments the partial enzymatic hydrolysis by α-L-arabinofuranosidase results in a loss of almost about 50% of the total Ara residues but does not affect the gelling ability of the polysaccharide.

In another aspect of the invention a laxative polysaccharide comprising an arabinoxylan is provided, the polysachharide containing at least about 20% arabinose and 70% xylose. The laxative polysaccharide is partially susceptible to acid hydrolysis (e.g. 0.005M sulfuric acid at 95 C) but not substantially susceptible to fermentation by intestinal microflora. Acid hydrolysis (0.005M sulfuric acid at 95 C) of the polysaccharide results in the release of some monsaccharides but no oligosaccharides, such that after 2 hours of acid hydrolysis the composition of the remaining partially-digested polysaccharide was less about 20 percent arabinose and greater than about 75% xylose; and after 6 hours of acid hydrolysis, the remaining partially-digested polysaccharide comprises less than about 15% arabinose and greater than about 80% xylose. Preferred polysaccharides are predominantly xylose, as indicated above, yet acid hydrolysis for two hours releases about 26% of the arabinose and only 8.5% of the xylose residues. After 6 hours of acid hydrolyis, at least about 57% of the arabinose and only 18% of the xylose is released, notwithstanding that at each time point the absolute amount of arabinose and xylose were about equal

The laxative polysaccharide is also partially susceptible to enzymatic hydrolysis by α-L-arabinofuranosidase. In preferred embodiments, the laxative effects of the polysaccharide are retained even after partial hydrolysis, for example, by enzymes.

The laxative polysaccharide can be readily isolated from plants, in particular Plantago. In preferred embodiments, the laxative polysaccharide is isolated from Plantago ovata. Still more preferred are polysaccharides isolated from Plantago ovata Forsk.

The structure of the laxative polysaccharide preferably comprises a backbone structure of β 1,4 linked xylopyranose residues. At least 50% of the backbone xylose residues have branches at the O-2 and O-3 positions. The branching structures preferably comprise xylose, or an arabinoxylan trisaccharide. In preferred embodiments the trisaccharide is Ara-Xyl-Ara. In more highly preferred embodiments, the trisaccharide is Ara(α1→3)-Xyl(β1→3)-Ara(α1→, and even more preferably L-Araf(α1→3)-D-Xylp(β1→3)-L-Araf(α1→. Substantially all of the xylose residues in the polysaccharide are in the pyranose ring form and substantially all of the arabinose residues are in the furanose ring form.

In preferred embodiments, the highly branched polysaccharide has greater than 50% of its O-2 or O-3 backbone positions substituted with xylose or the trisaccharide. In more preferred embodiments at least about 60, 70, 75, 80, 81, 82, 83, 84 or 85% of the O-2 or O-3 positions are branching points comprising either an xylose residue or a Ara-Xyl-Ara trisaccharide. In more preferred embodiments, the trisaccharide branch has the structure Ara(α1→3)-Xyl(β1→3)-Ara(α1→, and more preferably still, L-Araf(α1→3)-D-Xylp(β1→3)-L-Araf(α1→.

In presently preferred embodiments, approximately one third of the total glycosyl residues in the polysaccharide are in nonreducing terminal positions. Also preferred are polysaccharides in which the proportion of unsubstituted (→4) xylose is relatively small.

In preferred embodiments, the laxative effects of the polysaccharide are complemented by the cholesterol reducing effects of the polysaccharide.

The following examples are provided to describe various embodiments and aspects of the invention in greater detail. They are intended to further illustrate, not to limit, aspects of the invention described herein.

### EXAMPLE 1

### Fractionation of Psyllium Seed Husks

Milled psyllium seed husks (2 g) were stirred with 0.2 N potassium hydroxide (400 ml) containing sodium borohydride (400 mg) in a nitrogen atmosphere for 90 minutes. The mixture was centrifuged for 20 minutes at 23,500×g. The supernatant was decanted from an insoluble fraction that had settled out in the centrifuige bottle. The insoluble fraction (designated “Fraction A”) was stirred with fresh potassium hydroxide-sodium borohydride solution (100 ml) for an additional 15 minutes and re-centrifuged. The supernatants from both centrifugations were combined. The pH of the combined supernatants was adjusted to 4.5 with glacial acetic acid, with stirring, at ambient temperature, then centrifuged for 60 minutes at 25,500×g. The supernatant (Fraction C) was decanted from a gel mass (Fraction B) that had settled out in the centrifuge bottle. The gel was washed gently with water (50 ml) to remove adhering supernatant solution and this wash water was added to the supernatant. The gel fraction was desiccated by treatment with 95% ethanol and finally washed with diethyl ether and dried.

Prior to desiccating Fraction B, in some instances the fraction was further purified by repeating the alkaline solubilization and acidification steps described above. The gel was resuspended in 0.2 N KOH, acidified to pH 4.5 and centrifuged to recover the gel.

### EXAMPLE 2

### Measurement of the Sugar Composition of Psyllium Seed Husk Fractions B and C

The respective sugar compositions of the gel fraction (Fraction B) and the acid soluble fraction (Fraction C) produced by the method set forth in Example 1 were determined. Results are shown in Table 1.

TABLE 1
Composition of psyllium seed husk fractions (dry wt).

| | Psyllium | | | |
|---|---|---|---|---|
| | Seed | Alkali | Alkali Soluble | |
| | Husks | Insoluble | Acid Insoluble | Acid Soluble |
| | (2 g) | (Fraction A) | (Fraction B) | (Fraction C) |
| Yield (g) | — | 0.328 | 1.164 | 0.255 |
| Composition | | | | |
| (% dry wt) | | | | |
| Rhamnose | 3.17 | 0.49 | 0.95 | 14.86 |
| Arabinose | 19.93 | 33.58 | 19.45 | 8.11 |
| Xylose | 49.15 | 3.16 | 67.20 | 38.79 |
| Mannose | 2.17 | 10.84 | 0.0 | 0.0 |
| Galactose | 3.83 | 12.86 | 1.59 | 1.89 |
| Glucose | 4.37 | 19.34 | 0.37 | 0.75 |
| Uronic Acids | 5.41 | 3.58 | 1.74 | 16.84 |
| Ratios of | | | | |
| % dry wts | | | | |
| Xylose/ | 15.50 | 6.45 | 70.70 | 2.61 |
| Rhamnose | | | | |
| Xylose/ | 2.47 | 0.094 | 3.46 | 4.78 |
| Arabinose | | | | |
| Xylose/ | 12.83 | 0.25 | 42.26 | 20.52 |
| Galactose | | | | |
| Xylose/ | 9.08 | 0.88 | 38.62 | 2.30 |
| Uronic | | | | |
| Acids | | | | |

### EXAMPLE 3

### Measurement of the Viscosity of the Psyllium Seed Husk Gel-Forming Fraction

Approximate relative viscosity of the gel-forming fraction (Fraction B) of psyllium husk was determined. The solution for the relative viscosity determination was prepared by stirring overnight a 0.2% solution of the dry fraction in formamide. The solution was then heated with stirring to 70° C. over a 5 minute period, and stirred for. 1 hour at ambient temperature. Efflux times was measured using a pipet viscometer (Ostwald Dropping Pipet Viscometer, Cat. No. 13-695, Fisher Scientific, Pittsburgh, Pa.). The efflux time mesurements were repeateded three times and mean values determined.

TABLE 2
Apparent viscosity of psyllium seed husk Fraction B.

| | Efflux Time (sec) |
|---|---|
| Alkaline Soluble, Acid Insoluble | 876 |
| Fraction | |
| (Fraction B) | |
| Formamide (solvent only) | 303 |

The approximate relative viscosity is 2.89.

### EXAMPLE 4

### Solvent Extraction of Psyllium Seed Husk Components

Psyllium seed husk components may also be obtained by extraction with various solvents. Such solvents include formamide, dimethylsulfoxide and 4-methylmorpholine N-oxide (50% solution in water).

Milled psyllium seed husk (2 g) was added in small portions to a selected solvent (200 ml) with stirring over 30-60 min. In one protocol, the mixture was stirred for two days at room temperature, then centrifuged for 40 min at 27,000×g. In alternative protocols, the mixture was stirred at elevated temperatures, up to 60° C., whereupon the length of stirring could be reduced to as little as 12 hours.

The pelleted insoluble material was resuspended and stirred in additional solvent (50 ml) for 30 min, then re-centrifuged. The combined supernatants were added, with stirring, to 95% ethanol (5 volumes), bringing the final ethanol concentration to 80%. Methanol, isopropyl alcohol, acetone or similar solvents could be substituted for ethanol in this step. The precipitate was collected and washed with absolute ethanol, followed by diethyl ether, then dried.

The ethanol-insoluble precipitate that forms is similar in composition to the alkali-soluble material described in Examples 1-3. The sugar compositions of fractions obtained by the foregoing initial solvent extraction are shown in Table 3.

TABLE 3
Sugar compositions of fractions (% dry wt)

| | | 4-Methyl- | |
|---|---|---|---|
| | | morpholine | |
| | Formamide | N-oxide | Dimethyl sulfoxide |
| Rhamnose | 3.03 | 3.25 | 0.48 |
| Arabinose | 18.95 | 16.54 | 17.38 |
| Xylose | 65.56 | 57.47 | 65.33 |
| Mannose | 0.05 | 0.0 | 0.12 |
| Galactose | 1.54 | 1.50 | 1.64 |
| Glucose | 0.21 | 0.22 | 0.34 |
| Uronic Acids | 4.30 | 4.69 | 0.94 |

### EXAMPLE 5

### Effect of Psyllium Seed Husks and Fractions Thereof on Reabsorption of Bile Acids from the Small Intestines of Rats

One of the major sites for the hypocholesterolemic action of dietary fiber is in the lower small intestine (ileum), through its effect on bile acids. Bile acids function as emulsifiers in the small intestine to facilitate fat digestion and absorption. They are synthesized from cholesterol in the liver, stored in the gall bladder and secreted into the small intestine in response to eating. Bile acids are conserved through re-absorption in the ileum and recycling back to the liver through the blood. Food is digested and fluid absorbed as digesta moves distally in the intestine, such that in the lower third of the small bowel, the lumenal contents consist largely of indigestible material (i.e., fiber) and some fluid. It is now known that many of the viscous soluble fiber sources retain their viscosity during transit through the upper gastrointestinal tract, and essentially become concentrated in the lower gut. The soluble fiber effectively interferes with the re-absorption of bile acids in the ileum, the only site in the gut that has the necessary transport proteins for bile acids. Bile acids lost through stool are replaced by new synthesis in liver from blood cholesterol, which effectively lowers blood cholesterol levels.

Rats were surgically modified by removing the cecum and colon, and re-connecting the end of the ileum to the rectum. After a recovery period of 7-10 days, these animals secrete soft, formed material referred to as ileal excreta. Ileal excreta is preferred to stool for measuring the effect of a substance on absorption of bile acids, because bacterial transformation of bile acids in the large intestine can render up to half of them unidentifiable in stool.

Test meals were fed to groups of rats. Each meal was fed to four rats; the test meals contained 5% fiber as either (1) psyllium seed husk (PSH); (2) cellulose (as a control); or (3) the amount of a selected seed husk fraction (A, B or C), singly or combined, that would be present in 5% psyllium seed husks. The test meals also contained a non-absorbable marker so that the concentration of the marker in the ileal excreta could be used to calculate of the amount of the test meal that was present in the ileal excreta collected.

The results of the acute test meal study are shown in Table 4.

TABLE 4
Bile acids excreted after feeding test meals containing
various soluble fibers.

| | Bile acids excreted* |
|---|---|
| Test Meal | (μmol/g test meal) |
| Cellulose | 8.32 ± 2.42 |
| Unmilled PSH | 18.41 ± 2.61 |
| Fraction A | 9.07 ± 2.75 |
| Fraction B | 16.08 ± 3.20 |
| Fraction C | 13.76 ± 2.71 |
| Combined A, B, C | 15.65 ± 1.37 |

Mean ± SEM, n = 4 rats

An increase in bile acids excreted indicates a concomitant decrease in bile acids re-absorbed into the blood. An agent that prevents re-absorption of bile acids into the blood from the ileum is considered to have hypocholesterolemic properties. The results above demonstrate the hypocholesterolemic properties of psyllium seed husks and, more notably, of the conveniently-administrable gel-forming fraction, Fraction B, which was essentially equivalent to intact psyllium seed husks in hypocholesterolemic effect.

### EXAMPLE 6

### The Unfermented Gel Component of Psyllium Seed Husks Promotes Laxation as a Lubricant in Humans

In addition to increasing stool weight, a supplement of psyllium seed husks (PSH) produces a stool that is slick and gelatinous. In this example, we demonstrate that a gel fraction of psyllium escapes microbial fermentation and is responsible for these characteristics that enhance Taxation.

Materials and Methods:

Experimental design. The study consisted of 3 periods, a screening phase, the PSH period and the basal period. During the screening phase, subjects consumed 15 g/d of Metamucil7 (5g/meal) (Smooth Texture Metamucil7, The Procter & Gamble Co., Cincinnati, Ohio), along with their usual diet, for 12 days to provide an additional 8.8 g/d of dietary fiber. Subjects were asked to complete food intake records during d 9 12 to evaluate compliance with a protocol and to obtain more information about typical food intakes. Two stools were obtained from each subject during d 9 12 also to evaluate compliance with the protocol. During the next 7 days (d 13 19 of the study), subjects consumed a defined, low fiber diet, and with each meal, the PSH and a nonabsorbable marker (chromic sesquioxide, 200 mg/meal). For the next two weeks (d 20 33 of the study), subjects consumed their usual diet to allow all of the PSH to be excreted. For the 7 days (d 34 40 of the study) of the basal phase, the same controlled, low fiber diet and nonabsorbable marker, but no PSH supplement, were consumed. This experimental design was based on results of a preliminary study. It was observed in the preliminary study that 7 10 d of ingesting the supplement were necessary to achieve a high level of PSH excretion. Preliminary observations also revealed that it took 7 10 d from ingestion of the last test dose for all of the PSH to be excreted. Therefore, the PSH period for data collection followed the 12 d of PSH consumption that constituted the screening phase and a crossover experimental design was not employed.

Stools and qualitative bowel response, food intake and activity data were collected daily during both weeks of controlled diet. This study was approved by the College of Agricultural and Life Sciences Human Subjects Committee, University of Wisconsin Madison.

Subjects. Twenty one of the 33 individuals who responded to local advertisements were enrolled in the screening phase. Criteria for exclusion from the screening phase were: lactose intolerance, non omnivorous diet and unwillingness to follow a specified diet. Subject compliance, reliability, availability and attitude were evaluated during the screening phase, and 15 subjects (8 males and 7 females) were selected to participate in the study. Fourteen subjects completed the study. (Data from one subject who admitted to not providing all stools during the specified collection periods were deleted from the final analysis.) Subjects ranged in age from 18 30 yr (mean SE, 24 1 yr) and were of normal body weight for height (body mass index, 24.2 0.9).

Diet. All subjects consumed fixed diets during the PSH supplemented and basal phases consisting of foods provided to them as part of the study. Breakfast was consumed at home and consisted of Rice Krispies7 cereal, skim milk, orange juice, white bread, margarine or butter, and jelly. Lunch consisted of a sandwich, fresh fruit, and milk. The evening meal consisted of a meat, a starch source, salad and dessert. Subjects consumed prescribed amounts of the menu foods. The amounts of bread and milk varied with daily energy needs. Limited amounts of fiber free snacks and alcohol also were permitted. Coffee was allowed ad libitum. Subjects were supervised at the evening meal. Consumption of the supplement and nonabsorbable marker was verified daily when empty packets were exchanged for the next day's allotment of supplement.

Data and Sample Collection. All stools excreted during the PSH and basal phases of the study were individually collected, refrigerated promptly, and weighed and frozen within 8 h of collection. Subjects evaluated each stool using a 9 point rating scale consisting of: 4 disagree the most, 3 disagree extremely, 2 disagree very much, 1 disagree, 0 neutral, +1 agree, +2 agree very much, +3 agree extremely, and +4 agree the most possible.

Subjects completed daily food intake records to verify consumption of the items in the controlled diet. Physical activity forms were completed daily to identify any major changes in daily routine. Daily routines for each participant remained consistent throughout the two weeks of sample collection.

Analyses. Stools excreted during d 4 8 of each controlled diet period (d 16 20 and d 37 41 of the study) were thawed, pooled for each subject by hand mixing and re frozen until use or processed as outlined below. Duplicate aliquots (3 g) were dried (16 h, 70° C.) to determine moisture content (Marlett et al., JNCI 76: 1065-1070, 1986). Stool chromium content was determined using a modification (Hosig et al., Cereal Chem. 73: 392-398, 1996) of the method of Guncaga et al. (Clin. Chim. Acta 47: 77-81, 1974). Portions of pooled stool were lyophilized for duplicate (25 mg) determinations of neutral and amino sugar content by gas chromatography as the, alditol acetate derivatives after acid hydrolysis and reduction (Kraus et al., J. Chromatog. 513: 71-81, 1990; Monsma et al., Appl. Environ. Microbiol. 58: 3330-3336, 1992) and for uronic acid analysis using a colorimetric assay (Blumenkranz et al., Anal. Biochem. 54: 484-489, 1973). An aqueous extract of thawed, pooled stool was used to determine the relative viscosity of stool (Ostwald dropping pipet viscometer, Cat. No. 13 695, Fisher Scientific, Pittsburgh, Pa.). Aqueous fractions were obtained by vortexing aliquots (2 g) with water (10 ml), centrifuging (30,000×g, 30 min, 4° C.), and recovering and re centrifuging the supernatant.

The fractionation procedure to isolate the component responsible for the gel characteristic was applied to thawed aliquots of pooled stool from each subject from both diet periods. Aliquots (25 g) were de lipidated and subjected to base (0.18 N KOH) containing sodium borohydride (0.026 M) to minimize base catalyzed depolymerization. The alkali soluble fraction was acidified to pH 4.5 with glacial acetic acid and a precipitate (Fraction 1) recovered by addition to ethanol. Fraction 1 was suspended in water, heated to boiling, centrifuged and the supernatant reduced in volume by roto evaporation. The concentrate was added to ethanol to give a final alcohol concentration of 70%. The fibrous mass which formed by addition of the concentrated supernatant to ethanol was washed with ethanol, ether and vacuum dried. This material represented the gelatinous component of PSH stools (Fraction 2). No precipitate formed at this point during the analysis of the control stool samples. Neutral and amino sugar contents and uronic acid concentrations were determined on aliquots of this material, as outlined above.

Macronutrient and energy contents of the planned menus and of the daily intakes of each subject were calculated using nutrient composition tables. Dietary fiber intakes were calculated from food intake records using a detailed database of the fiber content and composition for US foods. The sums of the daily intakes on d 2-6 of each controlled diet period (d 14-18 and d 35-39) were used as the intake of the neutral sugars (glucose, arabinose, xylose, mannose, galactose) and the uronic acids for the determination of apparent digestibility of fiber derived sugars. The sugar composition of the PSH supplement also was measured by gas chromatography after acid hydrolysis, as outlined above. Fecal excretion of sugars was adjusted to reflect excretion of 5 d of intake using the content of chromium in the pooled stool (Chen et al., Am. J. Clin. Nutr. 68: 711-719, 1998). Apparent digestibilities of fiber derived sugars were calculated as the difference between intake and excretion, and expressed as a percentage of intake (Chen et al., 1998, supra).

Statistical Analyses. Data are reported as the mean SE. Data collected during the basal and psyllium supplemented phases of the study were compared by one way analysis of variance using SAS computer program software release 6.12. Significant differences were identified by the least significant difference means separation test.

Results: The first fraction (Fraction 1) recovered by the gel isolation scheme was extracted from both basal and PSH containing stools; this alkali soluble fraction from PSH stools was significantly larger (P<0.0005) than that extracted from basal excreta. An ethanol precipitable fraction (Fraction 2) that was gelatinous was extracted by hot aqueous treatment of the alkali soluble fraction from stool of subjects consuming the psyllium supplement. No gelatinous fraction was extracted from stool collected during the basal, low fiber phase of the study.

The major component in Fraction 2 isolated from stool of the 14 subjects consuming PSH was a polysaccharide that contained 763±18 mg of sugar/g of Fraction 2, most of which was xylose (64±1%) and arabinose (27±0%); the remainder of the sugars was (%): 2 glucose, 3 galactose, and 3 other sugars (fucose, ribose, mannose, myoinositol, muramic acid, glucosamine and galactosamine). The xylose and arabinose in this gel fraction accounted for 53.9±2.1 and 31.1±1.4% of the respective sugar in feces.

Since no fraction was obtained from basal excreta comparable to the gel isolated from the PSH containing stool, aqueous extracts of stool were prepared for a comparison of the relative viscosity of stool from the two study periods. The apparent viscosity of the aqueous extract of the PSH containing stool was significantly greater (p<0.01) than that of the basal, low fiber excreta, 238±38 vs. 128±7 sec.

The PSH supplement increased mean daily wet output from 117±7 to 188±13 g/d (P<0.0001), mean daily dry output from 29±2 to 37±2 g/d (P<0.0001), and stool moisture from 74.4±0.9% to 80.2±1.0% (P<0.05). Other measures of large bowel function also were significantly different. The mean wet weight of each stool increased from 121±6 to 173±14 g (P<0.005), mean dry weight of each stool from 30±2 to 34±3 g (P<0.0001), and defecation frequency from 1.0±0.1 to 1.1±0.1 (P<0.05) when PSH supplement was consumed.

The mixed food controlled diet contained a mixture of fiber derived sugars and the apparent digestibility of the fiber derived total neutral sugars was 67±4% during the low fiber basal period. The composition of the PSH preparation was (mg/g): 21 rhamnose, 127 arabinose, 325 xylose, 8 mannose, 25 galactose, 44 glucose, 35 uronic acids, 19 ash, 18 crude protein and 346 coating. Apparent digestibilities of the fiber derived xylose and arabinose decreased (P<0.001), while uronic acid digestibility increased (P<0.03) when the supplement was ingested. Subtracting xylose and arabinose excreted during the basal diet period from the amounts of these sugars in the PSH containing stool provides a means of estimating the apparent digestibility of the two major sugars in PSH. The apparent digestibilities of the xylose, arabinose and total neutral sugars provided by PSH were 59±5, 28±7 and 54±9%, respectively and were variable.

The test dose of PSH was well tolerated by all subjects. Compared to large bowel function during the basal period, PSH resulted in gentler bowel movements, softer stools that were easier to pass, greater ease of wiping, a feeling a complete relief, and increased bulk. The PSH supplement had no effect on abdominal cramping, an urge to defecate, or experience a repeat bowel evacuation, nor did it cause diarrhea, although subjects perceived more flatulence and bloating. Although soft and formed, most stools were visibly gelatinous and would vibrate when agitated. The macronutrient and dietary fiber contents provided by the foods in the diet did not change when the PSH supplement was consumed.

Thus, in contrast to other viscous fibers that are completely fermented in the colon, a component of psyllium is not fermented. The results of the foregoing study indicate that, by functioning as an emollient and a lubricant, the unfermented gel isolated from PSH-containing stool represents a new mechanism of laxation for a dietary fiber.

### EXAMPLE 7

### Demonstration of Poor Fermentation of the Gel-Forming Fraction of Psyllium Seed Husks In Vitro

In vitro fermentations of psyllium seed husks and its fractions were conducted in 40 ml fermentation flasks. Carbohydrate for fermentation was 200 mg, of which 31 mg was from veal infusion broth and 45 mg was from yeast; the remainder was from Fractions A, B and C of psyllium seed husks. Fractions were isolated from ground psyllium seed husks as described above. The “3 fractions combined” sample consisted of the three fractions isolated from the psyllium husk re-combined in proportion to their original concentration in the husks. Inoculum consisted of cecal contents harvested from rats fed purified diets containing 5% (by weight) psyllium seed husks. Experimental protocols and analyses were according to the methods described by Monsma & Marlett (J. Nutr. 126: 554-563, 1996). Results generated by this methodology have been shown to be similar to results obtained by in vivo fermentation in humans (Monsma et al., J. Nutr. 130: 585-593, 2000). All fermentations were prepared and conducted in an anaerobic chamber, and were performed in duplicate.

Table 5 shows the results of the in vitro fermentation. Carbohydrate in the fermentation flasks consisted of eleven neutral and amino sugars, along with uronic acids; these were provided by the test substrate veal infusion broth and yeast (see Table 5 footnotes “a” and “b”). The apparent fermentation of the sugars from these sources is summarized such that the fermentation of the test fractions is highlighted. As can be seen from the table, Fraction C was almost completely fermented by 24 hours. By contrast, Fraction B was only partially fermented (approx. 25-35%), and its fermentation appeared to have ceased by 48 hours. Fraction A was poorly fermented, and its fermentation was limited to the first 12 hours of the fermentation period.

TABLE 5
Disappearance of neutral sugars during in vitro
fermentation of psyllium seed husk and its fractions.

| | 0 h | | | | | |
|---|---|---|---|---|---|---|
| | buffer^{a} fract^{b} | | 12 h | 24 h | 48 h | 72 h |
| Polysaccharide/sugar | mg/flask | | % remaining | | | |
| Psyllium husk | | | | | | |
| rhamnose | 0.6 | 4.5 | 21 | 22 | 21 | 22 |
| arabinose | 0.9 | 28.1 | 84 | 84 | 76 | 82 |
| xylose | 2.6 | 69.3 | 68 | 46 | 39 | 40 |
| galactose | 4.3 | 5.4 | 61 | 67 | 61 | 66 |
| others^{c} | 65.5 | 9.2 | 23 | 22 | 19 | 21 |
| 3 fractions combined | | | | | | |
| Rha | 0.6 | 4.4 | 19 | 22 | 22 | 22 |
| Ara | 0.9 | 29.9 | 85 | 81 | 76 | 80 |
| Xyl | 2.6 | 70.0 | 73 | 47 | 44 | 44 |
| Gal | 4.3 | 5.7 | 56 | 61 | 58 | 60 |
| others | 65.5 | 8.6 | 24 | 22 | 21 | 20 |
| Fraction C | | | | | | |
| Rha | 0.6 | 21.4 | 16 | 6 | 6 | 6 |
| Ara | 0.9 | 11.6 | 47 | 19 | 18 | 19 |
| Xyl | 2.6 | 57.3 | 53 | 11 | 9 | 8 |
| uronic acids | 3.1 | 30.5 | 22 | tr | tr | tr |
| others | 69.9 | 3.6 | 14 | 12 | 8 | 9 |
| Fraction B | | | | | | |
| Rha | 0.6 | 1.3 | 79 | 61 | 52 | 53 |
| Ara | 0.9 | 26.8 | 93 | 85 | 78 | 76 |
| Xyl | 2.6 | 91.4 | 84 | 76 | 65 | 59 |
| others | 69.9 | 2.2 | 13 | 10 | 10 | 10 |
| Fraction A | | | | | | |
| Ara | 0.9 | 49.2 | 100 | 100 | 100 | 100 |
| Xyl | 2.6 | 4.6 | 68 | 58 | 59 | 59 |
| Man | 15.4 | 16.0 | 56 | 58 | 65 | 55 |
| Gal | 4.3 | 19.8 | 91 | 89 | 94 | 98 |
| Glc | 35.3 | 29.4 | 53 | 50 | 54 | 55 |
| others | 17.4 | 0.7 | 27 | 23 | 19 | 19 |

^{a}Sugars in the veal infusion broth and yeast that are part of the fermentate and contributed about 70-75 mg of carbohydrate to the fermentation.
^{b}Sugars in the test substrate, calculated to contribute about 120-125 mg to the fermentation.
^{c}Includes those sugars from the following list that are not specifically listed: rhamnose, fucose, ribose, arabinse, xylose, mannose, galactose, glucose, myoinositol, glucosamine and galactosamine. Although uronic acids were included in the calculation of the amount of carbohydrate to be fermented, they have not been measured in all samples and therefore are not included in this summary, except for the Fraction C summary.

### EXAMPLE 8

### Further Characterization of the Gel-Forming Polysaccharide by Partial Hydrolysis

Experimental:

Materials

Anion exchange resin AG3-X4, 100-200 mesh and cation exchange resin AG50W-X8, 100-200 mesh were from the BioRad Corp, Richmond, Calif. α-L-Arabinofuranosidase (EC 3.2.1.55) from A. niger was obtained from Megazyme, Wicklow, Ireland. Psyllium seed husk, identical with the principal component of the preparation marketed as Metamucil®, was provided by the Procter & Gamble Company, Cincinnati, Ohio.

Initial Preparation

The procedure for the isolation of the gel-forming polysaccharide (fraction B) and two additional fractions (A and C) from the husk are recorded elsewhere. (11) Fraction A amounted to 17%, B 57.5% and C 12.9% of the weight of the husk. (7) Thus, the three fractions account for nearly all of the carbohydrate and about 90% of the mass of the starting material. Neutral sugars in the husk and fractions were measured by GLC as alditol acetate derivatives using the method of Kraus et al., (12) as modified. (13) Uronic acids were measured by a colorimetric assay employing 3 phenylphenol (3-hydroxybiphenyl). (14) The results of these analyses were reported in a paper (7).

Purified fraction B was used for all structural studies. It was prepared by subjecting isolated fraction B sequentially to two further rounds of alkali solubilization followed by acidification, centrifugation, and dehydration, as in the original isolation procedure. The monosaccharide composition of this purified material appears in Table 7.

Partial Hydrolysis

Acid Hydroylsis: Two dry samples (50 mg) of fraction B were triturated with successive 0.3 ml portions of 0.005 M sulfuric acid to hydrate them, after which acid was added to final volumes of 10 ml. The samples were placed in a heating block at 95° C. and vortexed periodically to facilitate solution of the gel. One sample was removed from the heat source after 2 h and the other after 6 h. When the cooled solutions were examined in a Ubbelohde capillary viscometer, the observed efflux times were 161 sec for the 2 h sample, 108 sec for the 6 h sample, and 93 sec for the acid solvent. After measurement, the solutions were poured into ethanol to a final alcohol concentration of 80%, giving precipitates that could be recovered by centrifugation. These were washed with 80% ethanol, 95% ethanol, and ether, then dried for 2 d at 40 C. The yields of partially hydrolyzed products, B—H+, were 41 and 33 mg, respectively. When 0.17% solutions of the products were dialyzed against water, using a membrane having a cutoff at MW 6000-8000, B—H+(2 h) was 100% retained, and B—H+(6 h) was 95% retained. On examination by paper chromatography the supernatants from the alcohol precipitations were found to contain both arabinose and xylose; no oligosaccharides were detected. From the compositions of B H+(2 h) and (6 h), recorded in Table 7, and the weights of these fractions, it could be calculated that the amounts of monosaccharides released were: after 2 hours heating, Ara 3.6 mg, Xyl 5.3 mg; after 6 h, Ara 8.4 mg, Xyl 9.7 mg.

Isolation and characterization of a disaccharide. Sixteen 50 mg. samples of fraction B were triturated with 0.125 M sulfiric acid, acid was added to a final volume of5 mL per sample, and all were heated at 100° C. for 45 min. (15) The samples were combined and the solution was neutralized with barium carbonate, then filtered. The filtrate was concentrated in vacuo and passed through AG50W-X8 cation exchange resin. The effluent was reduced in volume to 20 mL and 95% ethanol was added to a final concentration of 79%. The resulting precipitate was recovered by centrifugation and the supernatant concentrated to a syrup. On analytical paper chromatography using butanol-benzene-pyridine-water (5:1:3:3, upper phase) as the solvent15 and ammoniacal silver nitrate for detection, no mobile components were found in the precipitate, but the syrup yielded three major spots corresponding to xylose, arabinose, and a substance having the mobility (RGlc 0.84) expected of a disaccharide. To isolate this unknown the main portion of the syrup was chromatographed on several sheets of Fisher thick paper and the relevant areas were excised. The compound was eluted with water and recovered by lyophilization.

Characterization of the unknown product was accomplished by sugar analysis and by NMR spectroscopic examination. For analysis, a portion (15 mg) was first reduced with sodium borohydride (20 mg) overnight, excess borohydride was destroyed by the addition of acetic acid, and the solution was evaporated to dryness. A sample of the reduced material was subjected to the acid hydrolysis step of the usual GLC sugar assay (see above), after the addition of myo-inositol as an internal standard. The NaBH4 reduction step of the assay was omitted; instead sodium acetate was added and the sample was dried and acetylated. A mixture of D-xylose and L-arabinitol was treated in the same manner. Like the authentic mixture, the reduced sample gave peaks corresponding to the acetates of α-xylopyranose, β-xylopyranose, and arabinitol. From the peak areas the ratio of xylose (α+β) to arabinitol was found to be 0.98, showing that the R_{Glc}0.84 product must be a xylosylarabinose.

Enzyme hydrolysis. Dry fraction B (0.5 g) was wetted by the careful dropwise addition of 5 mL of sodium acetate buffer (0.05 M, pH 4.0). The resulting gel was suspended in additional buffer (95 mL) and a-L-arabinofuranosidase (150 units in 3.2 M ammonium sulfate) was added. The gel suspension was shaken at 40° C. for 30 h, with further additions of 150 units of enzyme after 6 and 24 h. At the end of the incubation the gel mass was extremely well dispersed, giving the mixture a translucent appearance. The mixture was centrifuged for 20 min at 23,500×g and the gel pellet (fraction B-E1) was washed with 80% ethanol, 95% ethanol, absolute ethanol, and ether before drying at 40° C. (yield 282 mg). The supernatant was deionized by sequential passage over cation and anion exchange resins, concentrated, and poured into ethanol to a final alcohol concentration of 70%. The precipitate (Fraction B-E2) was recovered, washed, and dried (118 mg). The compositions of fractions B E1 and B-E2 are given in Table 7.

Results of Hydrolyis Experiments:

Compositional analysis and characterization of hydrolysis products:

As noted above, careful examination showed that both arabinose and xylose residues were cleaved from purified fraction B by mild acid treatment. According to the calculations outlined herein, about 29% of the arabinose and 13% of the xylose residues were lost during the first 2 hours, while after 6 hours the figures were ˜66% and ˜23%, respectively. The absolute amounts of xylose released were nearly the same as for the arabinose, reflecting the predominance of the former sugar in the starting polysaccharide.

NMR spectra were used to complete the characterization of the disaccharide fragment obtained from fraction B by brief heating with moderately strong acid. As already described, compositional analysis showed the product to be a disaccharide of xylose and arabinose having the sequence Xyl→Ara. Since earlier work (3,4) established the xylose of psyllium polysaccharides as the D-isomer, and the arabinose as the L-form, it was assumed these were the isomers present in this fragment. The proton spectrum of the disaccharide in D₂O revealed the presence of two components in a 68:32 ratio. The vicinal H—H coupling constants derived from the spectrum (Table 8) show that the arabinose residue, in the pyranose ring form, adopts the alternate chair conformation (⁴C₁ for L-sugars) in both components. Thus, in the major component, where J_{1,2} for Ara H−1=7.5 Hz, the arabinose unit is the α-anomer, and in the minor component (J_{1,2}2.4 Hz) the β-anomer. The β-anomeric configuration of the xylose residue is indicated by the value 8.1 Hz for J_{1,2} shown by both species of the free disaccharide, and 7.7 Hz found for the derived alditol.

The 1→3 location of the interunit linkage was established by the 13C spectrum, where the signals for Ara C-3 in the α- and β-anomers were found at δ 82.6 and 79.1, respectively, on the average ˜11 ppm downfield of the signals for C-2 and C-4. This sizable downward shift is a definitive indication of substitution at position 3; in unsubstituted arabinopyranose the differences between the δ values for C-3 and those for C-4 are confined to the range of 0-3.9 ppm. (22). Confirmation of our signal assignments, clearly distinguishing the disaccharide from its possible linkage isomers (β-Xyl attached to O-2 or O-5 of Ara, or α-Xyl at any position), was provided by comparison with literature data for the related compounds Galβ(1→3)Ara (for the arabinose moiety) (23) and the several xylobioses (for the xylose unit). (21) Thus the data as a whole identify the hydrolysis fragment as 3-O-β-D-xylopyranosyl-L-arabinose, a disaccharide previously characterized, without NMR analysis, as a hydrolysis product of the mucilage of Opuntia ficus-indica (prickly pear cactus). (24) The sequence D-Xylp(1→3)L-Ara has been found in a number of plant polysaccharides, with the xylose occurring in both the a-anomeric and β-anomeric forms. The α form is apparently the more common one.

An examination of fraction C from the crude psyllium extract was of interest because of earlier conflicting ideas (see Introduction) about the relationship between the uronic acid present in the extract and its major, arabinoxylan component. Our results show that uronic acid and the associated sugar rhamnose segregate in fraction C; gel-forming fraction B is essentially devoid of these two saccharides (Table 7). As noted in the Experimental, fraction C could be further separated into a detergent-precipitable polyuronide (C-2, 82% uronic acid+Rha) and a xylose-rich arabinoxylan (C-1). Interestingly, the latter formed a viscous solution in water, not a gel.

### Example 9

### Further Characterization of the Gel-Forming Polysaccharide by Methylation and Reductive Cleavage

Methylation and Reductive Cleavage Analyses:

The purified fraction B and its 2 h and 6 h hydrolysis products were methylated by a modification of the method of Ciucanu and Kerek. (16) The sample (3.2 mg) and a small stir bar were placed in a small flask and dried under high vacuum for 24 h. Dimethyl sulfoxide (4.0 ml) was then added and the mixture was kept overnight at room temperature to yield a clear solution. This was treated with powdered NaOH (60 mg) and the mixture was stirred for 2 h, then placed in a small bath sonicator overnight. Iodomethane (0.4 ml) was added, and after being stirred for 6 h the mixture was again placed in the sonicator overnight. Chloroform (5 ml) was added to the brown solution, the mixture was stirred for 30 min, water (1.5 ml) was added and stirring was continued for a few min, then after phase separation the water layer was removed. The chloroform solution was extracted six times with 4-ml portions of dilute Na₂S₂O₃ to remove brown color, three more times with water after adjustment to pH 4-6 with a few drops of 30% HOAc, and then dried over Na2SO4 and evaporated to dryness under a stream of nitrogen.

Reductive cleavage of the methylated polysaccharides was carried out in the presence of triethylsilane and a mixture of trimethylsilyl methanesulfonate and boron trifluoride etherate. (17) The products were acetylated in situ (17) and analyzed by GLC-MS on a DB-17 column programmed from 40 to 200° C. at 2° C./min. The identification of the resulting substituted anhydroalditols was accomplished by comparing them with synthetically prepared anhydroxylitol, (18) anhydroarabinitol, (19) and anhydrogalactitol (20) derivatives.

For traditional analysis the methylated polysaccharide was dissolved in 3.2 mL of chloroform and a 0.5 niL aliquot was removed and evaporated to dryness under a stream of dry nitrogen in a hydrolysis tube. Trifluoroacetic acid (0.5 mL of 4 M) was added and the tube, capped and evacuated, was kept 4 h at 105° C. After cooling the mixture was subjected to several cycles of isopropyl alcohol addition and evaporation (N₂ stream) to remove the trifluoroacetic acid, then the residue was dissolved in 1 mL of water. Sodium borodeuteride (10 mg) was added and the mixture was kept at room temperature overnight, then treated with 10 μL of glacial HOAc to destroy excess reagent and evaporated to dryness. Borate was removed by repeated additions and evaporations first of 9:1 methanol-acetic acid, then methanol alone. The residue was dissolved in 1.5 niL of water, the solution was deionized with a few beads of Amberlite IR-120(H₊), and after filtration again evaporated to dryness. Pyridine and acetic anhydride (0.4 mL each) were added, reaction was allowed to proceed overnight, and then the reagents were removed by evaporation under vacuum at 40° C. followed by two cycles of methanol addition and evaporation. The residue of partially methylated 1-deuteroalditol acetates was dissolved in 0.5 mL of chloroform and analyzed by GLC combined with CIMS and EIMS on a DB-5 column programmed from 80 to 180° C. at 1° C./min, followed by a hold at 180° C. for 30 min.

Results:

The application of reductive cleavage methodology to fraction B and its 2 h and 6 h hydrolysis products provided an unequivocal determination of the ring forms of the glycosyl units. (25,26) All the xylose residues yielded exclusively 1,5-anhydroxylitol derivatives, and the arabinose residues gave only 1,4-anhydroarabinitol derivatives. Hence xylose is present only in the pyranose ring form, while arabinose is solely in the furanose form.

Compositional data were obtained from our analyses of the reductive cleavage mixtures, but the values are unreliable because of the significant volatility of the anhydropentitol derivatives. For purposes of linkage analysis we therefore look to the figures provided by the quantitation of the methylated alditol acetate mixtures resulting from the standard methylation procedure. The identities of the individual alditol acetates were confirmed by one-to-one correlation with the components of the anhydroalditol mixture from reductive cleavage analysis. (For example, the only 2,3,5-trimethylated anhydroalditol in this mixture was shown to be an anhydroarabinitol. Therefore the 1,4-di-O-acetyl-2,3,5-tri-O-methylpentitol found among the products of standard methylation must be an arabinitol derivative.) Table 9 presents the data from the methylation and reductive cleavage analyses.

The highly branched nature of the purified polysaccharide is evident from the methylation analysis data. Some 34% of the glycosyl residues of fraction B are in nonreducing terminal (T) positions. Since it is inherent in polysaccharide structure that in a molecule having substantial branching the number (n) of branch point (BP) units approximately equals the number (n+1) of terminal units, one would expect about one third of the residues of Fraction B to be in branching positions. As may be seen from the table, the actual figure is 33%.

The methylation analysis of the partial acid hydrolyzates of fraction B gave unexpected results, in that the ratios of apparent terminal residues to apparent branch point units (T:BP) depart widely from the norm, reaching 1.5 for the 2 h sample and 1.7 for the 6 h. Independent methylation analyses of fraction B—H+(6 h), done in another laboratory, gave values agreeing within experimental error with the figures in Table 9.

At face value, these T:BP ratios >>1 indicate small numbers of branches per molecule, and if the molecule is ‘highly branched’ the number of ordinary interior residues (i) and thus the overall d.p. must also be small. Specifically, the values T:BP=1.5 and I (=% i)=27 suggest a d.p. in the heptasaccharide range (MW ˜950) for fraction B—H+(2 h), and T:BP=1.7, I=29 suggest the penta- to hexasaccharide range (MW ˜700-800) for the d.p. of fraction B—H+(6 h). These estimates are however inconsistent with the dialysis behavior of the two fractions, which were retained by membranes having MW cutoffs in the 6000-8000 range. Some of this discrepancy might be due to molecular association in solution, but the gap seems too large for sole attribution to this cause. The values in the B—H+(2 h) and B—H+(6 h) columns of Table 9 are thus subject to some question, but they may be regarded as indicative of trends in the way the linkage composition of fraction B changes during partial acid hydrolysis.

### Example 10

### Further Characterization of the Gel-Forming Polysaccharide by NMR Spectroscopy

NMR Spectroscopy

Samples (15-30 mg) of fraction B and its partial hydrolysis products in DMSO-d₆ or D₂O(400 μL) were examined in a Bruker DRX-360 instrument fitted with a 5-mm ¹H/broad band gradient probe with inverse geometry (proton coils closest to the sample). We used the standard Bruker implementations of the traditional suite of 1D and 2D (gradient-COSY, TOCSY, ¹H detected and gradient-selected HMQC, HMQC-TOCSY, HMBC) pulse sequences; only selected HMQC experiments, and the most informative HMQC-TOCSY, are shown and discussed here. The TOCSY mixing time was 80 ms; all other parameters were standard. It was not possible to prepare a D₂O solution of fraction B at a concentration sufficient for ¹³C NMR, so DMSO-d₆ was employed as the solvent for this fraction. The 6 h acid hydrolysis product proved to be adequately soluble in D₂O, and useful results were obtained with the solution. The solutions were extremely viscous at 300 K but were run at that temperature to permit comparison with chemical shift data from the literature. Relevant data for elevated temperatures are lacking. Referencing in DMSO-d₆ was from the solvent signals (δ_{H} 2.49 ppm; δ_{C} 39.5 ppm). For D₂O solutions, as is now common, acetone was used as the internal standard. The standard (2 μL) was added after acquisition of the required spectra and the reference back-applied to those spectra (δ_{H} 2.225 ppm; δ_{C} 31.07 ppm).

The usual pulse sequences were employed in measuring the spectra of the isolated disaccharide and its reduction product in D₂O with a Bruker 750 MHz instrument. Assignments were made with the aid of COSY, HSQC, and DEPT spectra. Proton chemical shifts and coupling constants were accurately determined by full spectral simulation using gNMR 3.6 for MacOS (Cherwell Scientific Publishing, Oxford, UK). The data are recorded in Table 8.

Results from NMR Spectroscopy:

Representative HMQC and HMQC-TOCSY spectra are shown in FIG. 1. In the plots each set of ¹H—¹³C correlation peaks (identified as such from the usefully redundant HMQC-TOCSY correlations) has a different color, and the major sets are further defined by grid lines.

Turning first to panel A of the figure, it will be seen that the HMQC spectrum of fraction B in DMSO is deceptively clean. It shows well resolved arabinofuranose peaks (blue), and a series of contours characteristic of xylopyranose residues, but lacks sufficient detail for linkage assignment. We suspect that the extreme viscosity of the solution and the consequently short t2 relaxation times artificially enhanced the signals of the more mobile terminal units over those of the presumably constrained internal units.

The spectrum of the fraction solubilized (B-E2) by the arabinofuranosidase treatment, shown in FIG. 1B, differs from that of the starting material only by the complete disappearance of the arabinofuranose peaks. Since it is known from the compositional analysis (Table 7) that fraction B-E2 still contains a significant percentage of arabinose residues, the absence of Ara peaks from the spectrum presumably reflects the removal of most or all of the terminal Ara residues by the enzyme.

The 6 h acid hydrolysis product, which formed a low-viscosity solution, gave more detailed spectra (HMQC, FIG. 1C; HMQC-TOCSY, FIG. 1D), and the fact that these were determined in D₂O allowed us to use reference data from the literature in analyzing them. In Table 10 chemical shifts measured in the HMQC-TOCSY experiment are given for the six discernible ¹H—¹³C correlation sets, along with comparable data from the literature indicating the identity and linkage arrangement of the glycosyl residues responsible for each pattern. Thus, the units having δ_{H}-1 5.40, δ_{C}-1 109.0 (cyan) can be identified as terminal α-arabinofuranose residues. Next, three sets of peaks have shifts characteristic of terminal δ-xylopyranose units: δ_{H}-1 4.72, δ_{C}1 104.6 (red);δ_{H}-1 4.81, δ_{C}-1 103.7 (purple); and δ_{H}-1 4.59, δ_{C}-1 103.8 (green). Units showing δ_{H} 1 4.75, δ_{C}-1 104.5 (dark blue) have shifts corresponding to those recorded for 3-linked βxylopyranose.[→3Xylpβ(1→] residues, and those with δ_{H}-1 4.68, δ_{C}-1 101.3 (magenta) can be identified with 2,4-linked, branching xylopyranose [→4[→2]Xylpβ(1→] units. In this case the ¹³C shift of the anomeric carbon does not match well with the literature value of 105.3 ppm, but when account is taken of the fact that the relevant residue in the literature model is a methyl glycoside this discrepancy can be tolerated.

In summary, the NMR data point to the presence, in molecules of psyllium fraction B and its partial hydrolysis products, of α-L-arabinofuranose end groups, and of β-D-xylopyranbse residues in terminal, 3-linked interior, and 2,4-linked branch point locations. These findings corroborate the results of our methylation analyses, discussed above. In one respect the NMR analysis extends the methylation results, revealing three sets of correlation peaks characteristic of terminal xylopyranose units, which suggests that the xylose end groups of fraction B—H+(6 h) occupy a corresponding number of distinct microenvironments. This in turn supports the notion of a complex, highly branched structure for the polysaccharide.

Methylation analysis is of course not capable of distinguishing among the different surroundings of residues that have the same linkage mode. However, it may be seen from Table 9 that our methylation studies reveal three types of sugar residues not found in our NMR experiments, namely 3-linked interior arabinofuranose units, 4-linked interior xylopyranose units, and 3,4-linked branch point xylopyranose units. Our characterization of xylopyranosyl(β1→3)arabinose as a degradation product validates the methylation finding in the case of 3-linked arabinose residues. In view of the fact, mentioned above, that an independent laboratory could fully confirm the methylation results we obtained on fraction B—H+(6 h), the argument for the additional xylose units also seems solid. At least one possible explanation for the lack of NMR evidence for the three sets of residues is the apparent response of only the more mobile components.

Conclusion:

Accepting the highly branched arabinoxylan nature of the polysaccharide, one can visualize a main chain of densely substituted 1,4-linked xylopyranose residues, some carrying single xylose units, others bearing trisaccharide branches having the structure Arafα(1→3)Xylpβ(1→3)Araf. The nearly equivalent mole percentages of T-Xyl and→2,4)Xyl [including “→2,3,4)Xyl”] residues (Table 9) suggest the attachment of the xylose single unit branches at position 2 of the underlying main chain residues. Similarly, the near equivalence of T-Ara, →3)Xyl, →3)Ara, and →3,4)Xyl is compatible with the proposed trisaccharide structure, and suggests the attachment of these branches at position 3 of the underlying main chain xyloses. Because of the dense branching of the structure the proportion of unsubstituted →4)Xyl is relatively small. The presence of Xylpβ(1→3)Ara sequences in the fraction B molecule is established by our isolation of the corresponding disaccharide as a hydrolysis product; the side chains would seem to be the most likely location for these sequences. Since the arabinose residues of the polysaccharide are in the furanose form the linkages of the T-Ara units to side-chain xylose and of the internal Ara units to the main chain would be labile to acid. Thus on partial acid hydrolysis one might expect to see an increase in the mol percent of T-Xyl at the expense of T-Ara, and if the linkage of the Ara Xyl Ara side chain is to O-3 of the underlying xylose there would be a conversion of →3,4)Xyl to →4)Xyl. Such trends are indeed discernible in the data of Table 3. Cleavage from the polymer by a arabinofuranosidase shows the T-Ara units to be α-anomers.

The structure outlined herein differs from that suggested for psyllium mucilage by Kennedy and coworkers (5,6) on the basis of their methylation analysis of the unfractionated husk. The analysis revealed a substantial content of →3)Xyl residues, which those authors assigned without explanation to the main chain of the xylan core. This assignment is not required by their data. A result consistent with our proposed structure was presented at a recent symposium by Edwards et al., (28) based on analysis of crude preparations from psyllium seed husk. These authors reported finding the tetrasaccharide L-Araf(1→3)[D-Xylp(1→4)]₂D-Xyl among the fragments produced by enzymic digestion of their extracts.

One desirable property of fraction B is its resistance to digestion by the intestinal microflora. This property suggests some unique features of molecular structure such as those described herein. Even assuming organisms of the flora possess enzymes capable of cleaving off the terminal α-L-arabinofuranose residues, the loss of these residues does not affect the gelling ability of the polysaccharide. Evidence to support this includes the experiments with arabinofuranosidase, in which the major portion of the substrate persisted as a gel after a prolonged incubation that removed almost 50% of the starting arabinose, and which reduced the T-Ara units to a level undetectable by NMR. Further digestion would require, for example, a complement of exoxylosidases and xylanases, which one would expect to be present in the gut. The resistance of the residual polysaccharide to the action of these enzymes must be a function of an unusual linkage pattern in the molecule and/or the high density of its branching.

References:
1. Cummings, J. H. The Effect of Dietary Fiber on Fecal Weight and Composition. In Dietary Fiber in Human Nutrition, 2nd ed., Spiller, G. A., Ed.; CRC Press, Boca Raton, Fla., 1993; pp. 263-349.
2. Anderson, J. W.; Allgood, L. D.; Lawrence, A.; Altringer, L. A.; Jerdack, G. R.; Hengehold, D. A.; Morel, J. G. Am. J Clin. Nutr., 2000, 71, 472-479.
3. Laidlaw, R. A.; Percival, E. G. V. J. Chem. Soc., 1949, 1600-1607.
4. Laidlaw, R. A.; Percival, E. G. V. J. Chem. Soc., 1950, 528-534.
5. Kennedy, J. F.; Sandhu, J. S.; Southgate, D. A. T. Carbohydr. Res., 1979, 75, 265-274.
6. Sandhu, J. S.; Hudson, G. J.; Kennedy, J. F. Carbohydr. Res., 1981, 93, 247-259.
7. Marlett, J. A.; Fischer, M. H. J. Nutr., 2002, 132, 2638-2643.
8. Marlett, J. A.; Kajs, T. M.; Fischer, M. H. Am. J. Clin. Nutr., 2000, 72, 784-789.
9. Marlett, J. A. Sites and Mechanisms for the Hypocholesterolemic Actions of Soluble Dietary Fiber Sources. In Dietary Fiber in Health and Disease, Kritchevsky D.; Bonfield, C., Eds.; Plenum Press, New York, N.Y., 1997; pp. 109-121.
10. Marteau, P.; Flourié, B.; Cherbut, C.; Corréze, J.-L.; Pellier, P.; Seylaz, J.; Rambaud, J.-C. Gut, 1994, 35, 1747-1752.
11. Marlett, J. A.; Fischer, M. H. U.S. Pat. No. 6,287,609; Chem. Abstr., 2000, 134, 32942; SciFinder Scholar, AN 2000:880972.
12. Kraus, R. J.; Shinnick, F. L.; Marlett, J. A. J. Chromatog., 1990, 513, 71-81.
13. Monsma, D. J.; Marlett, J .A. J. Nutr., 1996, 126, 554-563.
14. Blumenkrantz, N.; Asboe-Hansen, G. Anal. Biochem., 1973, 54, 484-489.
15. Gowda, D. C.; Gowda, J. P.; Anjaneyalu, Y. V. Carbohydr. Res., 1982, 108, 261-267.
16. Ciucanu, I.; Kerek, F. Carbohydr. Res., 1984, 131, 209-217.
17. Jun, J.-G.; Gray, G. R. Carbohydr. Res., 1987, 163, 247-261.
18. Elvebak II, L. E.; Knowles, V.; Gray, G. R. Carbohydr. Res., 1997, 299, 143-149.
19. Elvebak II, L. E. Ph.D. Thesis, University of Minnesota, 1994.
20. Elvebak II, L. E.; Abbott, C.; Wall, S.; Gray, G. R. Carbohydr. Res., 1995, 269, 1-15.
21. Bock, K.; Pedersen, C.; Pedersen, H. Advan. Carbohydr. Chem. Biochem., 1984, 42, 193-225.
22. Vogt, D. C.; Jackson, G. E.; Stephen, A. M. Carbohydr. Res., 1992, 227, 371-374.
23. Gast, J. C.; Atalla, R. H.; McKelvey, R. D. Carbohydr. Res., 1980, 84, 137-146.
24. McGarvie, D.; Parolis, H. Carbohydr. Res., 1981, 94, 57-65.
25. Rolf, D.; Gray, G. R. J. Am. Chem. Soc., 1982, 104, 3539-3541.
26. Gruber, P. R.; Gray, G. R. Carbohydr. Res., 1990, 203, 79-90.
27. Colquhoun, I. J.; Ralet, M.-C.; Thibault, J.-F.; Faulds, C. B.; Williamson, G. Carbohydr. Res., 1994, 263, 243-256.

28. Edwards, S.; Chaplin, M. F.; Blackwood, A. D.; Dettmar, P. W. Proc. Nutr. Soc., 2003, 62, 217-222.

TABLE 7
Monosaccharide composition of selected
fractions and hydrolysis products

| | Compositions (mol %) | | | | | | |
|---|---|---|---|---|---|---|---|
| | | B-H⁺ | B-H⁺ | | | | |
| Monosaccharide | Purified B | (2 h) | (6 h) | B-E1 | B-E2 | C-1 | C-2 |
| Arabinose | 22.6 | 19.4 | 11.4 | 13.7 | 7.2 | 16.0 | 3.9 |
| Xylose | 74.6 | 78.7 | 87.0 | 84.8 | 91.5 | 79.9 | 11.9 |
| Galactose | 1.5 | 1.5 | 1.3 | 1.3 | 1.2 | 2.3 | 1.8 |
| Glucose | 0.3 | 0.2 | 0.2 | 0.2 | 0.1 | 0.9 | 0.6 |
| Rhamnose | 0.4 | 0.1 | 0.1 | 0 | 0 | 0.3 | 40.5 |
| Uronic acid | 0.7 | nd | nd | nd | nd | 0.6 | 41.4 |

nd = not determined

TABLE 8
NMR data for β-D-xylopyranosyl(1→3)-L-arabinose and its derived alditol

| δ (ppm) | Positions | | | | | | | | | |
|---|---|---|---|---|---|---|---|---|---|---|
| or | Xylose unit | | | | | | Arabinose unit | | | |
| J (Hz)^{a} | 5ax | 5eq | 4 | 3 | 2 | 1 | | 4 | 3 | 2 |

Xylpβ1→3Arapα

| | | | | | | | 5ax | 5eq | | | | 1 |
|---|---|---|---|---|---|---|---|---|---|---|---|---|
| δ_{H} | 3.29 | 3.94 | 3.61^{b} | 3.44 | 3.35 | 4.60^{c} | 3.66 | 3.88 | 4.10^{b} | 3.77 | 3.64 | 4.54^{c} |
| J_{n-1,n} | 10.2 | 5.5 | 9.9 | 9.0 | 8.1 | | 1.0 | 2.1 | 3.9 | 9.8 | 7.5 | |

| | | | | | | | | | | |
|---|---|---|---|---|---|---|---|---|---|---|
| | J_{gem} 11.3 | | | | | J_{gem} 13.6 | | | | |
| δ_{C} | 65.9 | 70.0 | 76.3 | 74.0 | 105.1 | 66.7 | 69.0 | 82.6 | 71.8 | 97.3 |

Xylpβ1→3Arapβ

| | | | | | | | | | | | | |
|---|---|---|---|---|---|---|---|---|---|---|---|---|
| δ_{H} | 3.30 | 3.93 | 3.61^{b} | 3.44 | 3.35 | 4.60^{c} | 3.64^{c} | 4.02 | 4.15 | 3.97 | 3.96 | 5.24^{c} |
| J_{n-1,n} | 10.2 | 5.5 | 9.9 | 9.0 | 8.1 | | | 0.8 | 2.7 | 7.5 | 2.4 | |

| | | | | | | | | | | |
|---|---|---|---|---|---|---|---|---|---|---|
| | J_{gem} 11.3 | | | | | J_{gem} 13.9 | | | | |
| δ_{C} | 65.9 | 70.0 | 76.3 | 74.0 | 105.0 | 62.9 | 69.3 | 79.1 | 68.2 | 93.3 |

Xylpβ1→3Ara-ol^{f}

| | | | | | | | 5ₐ | 5_{b} | | | | 1ₐ, 1_{b} |
|---|---|---|---|---|---|---|---|---|---|---|---|---|
| δ_{H} | 3.28 | 3.95 | 3.61^{b} | 3.42 | 3.31 | 4.40^{c} | 3.69 | 3.77 | 23.83^{d} | 3.81 | 3.97^{e} | 3.73^{c} |
| J_{n-1,n} | 10.6 | 5.2 | 9.3 | 9.4 | 7.7 | | 6.71 | 3.4 | 6.66 | 2.3 | 6.3 | |

| | J_{gem} 11.8 | | | | | J_{gem} 12.3 | | ⁴J_{2,4} 0.8 | | |
|---|---|---|---|---|---|---|---|---|---|---|
| δ_{C} | 65.6 | 69.7 | 76.0 | 73.9 | 104.4 | 63.4 | 71.7 | 79.6 | 71.74 | 63.0 |

^{a1}H signal multiplicities are dd except as indicated by footnote.
^{b}ddd.
^{c}d.
^{d}dddd.
^{e}tdd.
^{f}The spectra of the disaccharide alditol were simpler than those of the anomeric mixture, but complete assignment of the ¹H signals could only be done on a sample prepared by reduction with NaBD₄, which differentiated positions 1 and 2 of the Ara-ol moiety from positions 4 and 5.

TABLE 9
Glycosyl-linkage compositions of fractions B, B—H⁺(2h), and
B—H⁺(6h) as deduced from standard methylation and
reductive cleavage analysis

| | | Amounts present | | |
|---|---|---|---|---|
| | | (mole percent)^{a} | | |
| Residue, | Corresponding alditol and | Purified | B—H⁺ | B—H⁺ |
| Linkage | anhydroalditol derivatives | B | (2h) | (6h) |
| T-Araf | 1,4-O-Ac₂-2,3,5-O-Me₃-arabinitol | 12.6 | 14.9 | 7.9 |
| | 1,4-Anhydro-2,3,S-O-Me₃-arabinitol | 11.6 | 14.4 | 2.2 |
| T-Xylp | 1,5-O-Ac₂-2,3,4-O-Me₃-xylitol | 20.4 | 28.3 | 36.1 |
| | 1,5-Anhydro-2,3,4-O-Me₃-xylitol | 23.7 | 40.9 | 26.5 |
| →3)Araf | 1,3,4-O-Ac₃-2,5-O-Me₂-arabinitol | 12.6 | 10.0 | 7.2 |
| | 3-O-Ac-1,4-anhydro-2,5-O-Me₂-arabinitol | 13.1 | 12.6 5.6 | |
| →3)Xylp | 1,3,5-O-Ac₃-2,4-O-Me₂-xylitol | 14.4 | 11.6 | 9.7 |
| | 3-O-Ac-1,5-anhydro-2,4-O-Me₂-xylitol | 12.7 | 14.8 | 13.0 |
| →4)Xylp | 1,4,5-O-Ac₃-2,3-O-Me₂-xylitol | 5.8 | 5.3 | 12.1 |
| | 4-O-Ac-1,5-anhydro-2,3-O-Me₂-xylitol | 1.6 | 1.5 | 14.1 |
| →3,4)Xylp | 1,3,4,5-O-Ac₄-2-O-Me-xylitol^{b} | 12.8 | 9.7 | 6.6 |
| | 3,4-O-Ac₂-1,5-anhydro-2-O-Me-xylitol | 7.2 | 2.3 | 0.8 |
| →2,4)Xylp | 1,2,4,5-O-Ac₄-3-O-Me-xylitol | 17.4 | 16.8 | 17.3 |
| | 2,4-O-AC₂-1,5-anhydro-3-O-Me-xylitol | 27.8 | 10.6 | 32.2 |
| →2,3,4)Xylp | 1,2,3,4,5-O-Ac₅-xylitol | 2.9 | 2.4 | 2.0 |
| | 2,3,4-O-Ac₃-1,5-anhydroxylitol | 2.0 | 1.8 | 5.3 |
| T-Galp | 1,5-O-Ac₂-2,3,4,6-O-Me₄-galactitol | 1.1 | 1.0 | 1.1 |
| | 1,5-Anhydro-2,3,4,6-O-Me₄-galactitol | 0.3 | 1.1 | 0.3 |
| Nonbranching | [→3)Araf + →3)Xylp + →4)Xylp] | 32.8 | 26.9 | 29.0 |
| interior | | | | |
| T:BP^{c} | $\frac{[ T \text{-} Araf + T \text{-} Xylp + T \text{-} Galp ]}{[ \to 3 , 4 ) Xylp + \to 2 , 4 ) Xylp + \to 2 , 3 , 4 ) Xylp ]}$ | 1.0 | 1.5 | 1.7 |

^{a}See text regarding the unreliability of the values for the derivatized anhydroalditols.
^{b}The use of borodeuteride in the reduction to the alditol stage permitted the mass-spectroscopic identification of this component as the 2-O-methyl derivative. Without deuterium substitution it would be indistinguishable from the 4-O-methyl compound, except by a procedure capable of separating enantiomers.
^{c}BP = branch point residues. The mol % →2,3,4)Xylp is included without multiplication on the supposition that the figure may represent single branch point residues on which the lone free OH group escaped methylation.

TABLE 10
NMR chemical shifts shown by fraction B-H⁺(6 h) (FIG. 1D)
and the literature values used for residue identification

| Residue, | Chemical Shift (δ) | | | | | | |
|---|---|---|---|---|---|---|---|
| Literature Model | H-1 | C-1 | C-2 | C-3 | C-4 | C-5 | Ref. |
| T-Araf (cyan) | 5.40 | 109.0 | 82.0 | 77.4 | 85.0 | 61.8 | |
| L-Arafα(1→3)-2-O-R^{a}-L-Arafβ(1→ | 5.25 | 107.8 | 82.0 | 77.5 | 84.8 | 62.0 | 27 |
| T-Xylp (red) | 4.72 | 104.6 | 74.6 | 76.3 | 70.1 | 66.1 | |
| T-Xylp (purple) | 4.81 | 103.7 | 74.2 | 75.5 | 70.1 | 66.2 | |
| T-Xylp (green) | 4.59 | 103.8 | 73.7 | 76.4 | 70.0 | 66.0 | |
| D-Xylpβ(1→3)D-Xylpβ(1→OMe | | 104.8 | 74.6 | 76.9 | 70.4 | 66.4 | 21 |
| D-Xylpβ(1→4)D-Xylpβ(1→OMe | | 103.1 | 74.0 | 76.9 | 70.4 | 66.5 | 21 |
| →3)D-Xylpβ(1→ (dark blue) | 4.75 | 104.5 | ˜74.0 | 84.2 | 68.5 | 65.7 | |
| →3)D-Xylpβ(1→4)D-Xylβ(1→OMe | | 103.0 | 73.8 | 84.9 | 69.0 | 66.2 | 21 |
| →2[→4]D-Xylpβ(1→ (magenta) | 4.68 | 101.3 | 81.4 | 74.3 | 77.3 | 63.5 | |
| →2[→4]D-Xylpβ(1→OMe | | 105.3 | 81.9 | 75.0 | 77.9 | 63.9 | 21 |

^{a}R = trans-feruloyl

The present invention is not limited to the embodiments described and exemplified above, but is capable of variation and modification without departure from the scope of the appended claims.

1. A compound of Formula I comprising a backbone of β 1,4 linked xylopyranose residues wherein K¹, K², K³, and K⁴ are each independently selected from hydrogen, xylose, or a trisaccharide consisting of arabinose-xylose-arabinose; and Z¹ and Z² are each hydrogen.

2. The compound of claim 1 wherein K¹ and K² are each independently selected from hydrogen or xylose and K³ and K⁴ are each independently selected from hydrogen or a trisaccharide consisting of Arabinose-Xylose-Arabinose.

3. The compound of claim 1 wherein the trisaccharide has the structure Ara(α1→3)-Xyl(β1→3)-Ara(α1→.

4. The compound of claim 3 wherein the trisaccharide has the structure L-Araf(α1→3)-D-Xylp(β1→3)-L-Araf(α1→.

5. The compound of claim 1 wherein the arabinose residues are in a furanose ring form and the xylose residues are in a pyranose ring form.

6. The compound of claim 1 wherein at least 50% of the total K¹, K², K³, and K⁴ are each independently selected from xylose and the trisaccharide Ara-Xyl-Ara.

7. The compound of claim 1 wherein at 66% of the total K¹, K2, K³, and K⁴ are each independently selected from xylose and the trisaccharide Ara-Xyl-Ara.

8. The compound of claim 1 wherein at least 75% of the total K¹, K², K³, and K⁴ are each independently selected from xylose and the trisaccharide Ara-Xyl-Ara.

9. The compound of claim 1 wherein at least 80% of the total K¹, K², K³, and K⁴ are each independently selected from xylose and the trisaccharide Ara-Xyl-Ara.

10. The compound of claim 1 wherein about 85% of the total K¹, K², K³, and K⁴ are each independently selected from xylose and the trisaccharide Ara-Xyl-Ara.

11. The compound of claim 10 which forms a gel in water.

12. The compound of claim 11 which resists digestion by intestinal microflora as measured after in an in vitro fermentation simulating in vivo colon fermentation after feeding to humans or rats.

13. The compound of claim 1 comprising about at least about 70% xylose and 20% arabinose, and not exceeding 2% galactose.

14. The compound of claim 13 which forms a gel in water.

15. The compound of claim 14 which is resistant to digestion by intestinal microflora as measured in an in vitro fermentation simulating human colonic fermentation.

16. The compound of claim 15 isolated from a plant.

17. The compound of claim 16 wherein the plant is Plantago ovata.

18. The compound of claim 17 which has a laxative effect in humans.

19. The compound of claim 17 which has cholesterol-lowering activity in humans.

20. A pharmaceutical composition comprising the compound of claim 1.

21. A method of promoting Taxation in a patient, comprising administering to the patient a laxation-promoting amount of the pharmaceutical composition of claim 20.

22. A method of lowering cholesterol in a patient, comprising administering to the patient the pharmaceutical composition of claim 20, in an amount effective to lower the patient's cholesterol.

23. A gel-forming polysaccharide comprising a backbone structure of β 1,4 linked xylopyranose residues, said backbone structure having branching structures on at least 50% of the O-2 or O-3 positions of the xylose residues, said branching structures comprising xylose or a trisaccharide of Ara-Xyl-Ara.

24. The gel-forming polsaccharide of claim 23 comprising at least about 70% xylose and 20% arabinose.

25. The gel-forming polysaccharide of claim 24 wherein the xylose residues are in a pyranose form and the arabinose residues are in a furanose form.

26. The gel-forming polysaccharide of claim 25 wherein the trisaccharide has the structure Ara(α1→3)-Xyl(β1→3)-Ara(α1→3).

27. A pharmaceutical composition comprising the gel-forming polysaccharide of claim 23.

28. A method of promoting Taxation in a patient, comprising administering to the patient a laxation-promoting amount of the pharmaceutical composition of claim 27.

29. The method of claim 28, wherein the pharmaceutical composition comprises between about 2 and about 6 g, based on dry weight, of the gel-forming polysaccharide.

30. A method of lowering cholesterol in a patient, comprising administering to the patient the pharmaceutical composition of claim 27, in an amount effective to lower the patient's cholesterol.

31. The method of claim 30, wherein the pharmaceutical composition comprises between about 3 and about 7 g, based on dry weight, of the gel-forming polysaccharide.
